SEQUENCE LISTING (11 sequences)

SEQ ID NO: 1 - DNA, 381 bp, Artificial Sequence
  Anti-spike glycoprotein nanobody of a coronavirus
caggtgcagctgcaggaaagcggtggtggtctggtgcaggccggtggtagcctgcgtctgagctgtgccgccagcggtcgtacctttagcgaatatgccatgggttggtttcgtcaggccccgggtaaagaacgtgaatttgtggccaccattagctggagcggtggtagcacctattataccgatagcgtgaaaggtcgttttaccattagccgtgataatgccaaaaataccgtgtatctgcagatgaatagcctgaaaccggatgataccgccgtgtattattgtgccgccgccggtctgggtaccgtggtgagcgaatgggattatgattatgattattggggtcagggtacccaggtgaccgtgagcagcggtagc

SEQ ID NO: 2 - DNA, 429 bp, Artificial Sequence
  Anti-spike glycoprotein nanobody of a coronavirus
aagcttcatcaccatcaccatcacggtggtggtggtagccaggtgcagctgcaggaaagcggtggtggtctggtgcaggccggtggtagcctgcgtctgagctgtgccgccagcggtcgtacctttagcgaatatgccatgggttggtttcgtcaggccccgggtaaagaacgtgaatttgtggccaccattagctggagcggtggtagcacctattataccgatagcgtgaaaggtcgttttaccattagccgtgataatgccaaaaataccgtgtatctgcagatgaatagcctgaaaccggatgataccgccgtgtattattgtgccgccgccggtctgggtaccgtggtgagcgaatgggattatgattatgattattggggtcagggtacccaggtgaccgtgagcagcggtagctaaaagctt

SEQ ID NO: 3 - DNA, 354 bp, Artificial Sequence
  Anti-spike glycoprotein nanobody of a coronavirus
caggtgcagctggtggaaaccggcggcggcctggtgcagccgggcggcagcctgcgcctgagctgcgcggcgagcggctttacctttagcagcgtgtatatgaactgggtgcgccaggcgccgggcaaaggcccggaatgggtgagccgcattagcccgaacagcggcaacattggctataccgatagcgtgaaaggccgctttaccattagccgcgataacgcgaaaaacaccctgtatctgcagatgaacaacctgaaaccggaagataccgcgctgtattattgcgcgattggcctgaacctgagcagcagcagcgtgcgcggccagggcacccaggtgaccgtgagcagc

SEQ ID NO: 4 - DNA, 491 bp, Artificial Sequence
  Anti-spike glycoprotein nanobody of a coronavirus
aagcttgaaaacctgtacttccaaggtgaattccaggtgcagctggtggaaaccggcggcggcctggtgcagccgggcggcagcctgcgcctgagctgcgcggcgagcggctttacctttagcagcgtgtatatgaactgggtgcgccaggcgccgggcaaaggcccggaatgggtgagccgcattagcccgaacagcggcaacattggctataccgatagcgtgaaaggccgctttaccattagccgcgataacgcgaaaaacaccctgtatctgcagatgaacaacctgaaaccggaagataccgcgctgtattattgcgcgattggcctgaacctgagcagcagcagcgtgcgcggccagggcacccaggtgaccgtgagcagcggatccgaaaacctgtacttccaaggtgactacaaggacgatgacgataagtggagccatccgcagtttgagaaatctagaaagctttaacatatgactcgagt

SEQ ID NO: 5 - DNA, 5875 bp, Artificial Sequence
  Synthetic plasmid
gacgtcttgacagctagctcagtcctagggattgtgctagcaggtttaatcgaattgacgtctttacggctagctcagtcctaggtactatgctagcaggtttaatcgaattcaaaagatcttttaagaaggagatatacatatgattactcatggttgttatacccggacccggcacaagcataagctaaaaaaaacattgattatgcttagtgctggtttaggattgtttttttatgttaatcagaactcatttgcaaatggtgaaaattattttaaattgggttcggattcaaaactgttaactcatgatagctatcagaatcgccttttttatacgttgaaaactggtgaaactgttgccgatctttctaaatcgcaagatattaatttatcgacgatttggtcgttgaataagcatttatacagttctgaaagcgaaatgatgaaggccgcgcctggtcagcagatcattttgccactcaaaaaacttccctttgaatacagtgcactaccacttttaggttcggcacctcttgttgctgcgggtggtgttgctggtcacacgaataaactgactaaaatgtccccggacgtgaccaaaagcaacatgaccgatgacaaggcattaaattatgcggcacaacaggcggcgagtctcggtagccagcttcagtcgcgatctctgaacggcgattacgcgaaagataccgctcttggtatcgctggtaaccaggcttcgtcacagttgcaggcctggttacaacattatggaacggcagaggttaatctgcaaagtggtaataactttgacggtagttcactggacttcttattaccgttctatgattccgaaaaaatgctggcatttggtcaggtcggagcgcgttacattgactcccgctttacggcaaatttaggtgcgggtcagcgttttttccttcctgcaaacatgttgggctataacgtcttcattgatcaggatttttctggtgataatacccgtttaggtattggtggcgaatactggcgagactatttcaaaagtagcgttaacggctatttccgcatgagcggctggcatgagtcatacaataagaaagactatgatgagcgcccagcaaatggcttcgatatccgttttaatggctatctaccgtcatatccggcattaggcgccaagctgatatatgagcagtattatggtgataatgttgctttgtttaattctgataagctgcaatcgaatcctggtgcggcgaccgttggtgtaaactatactccgattcctctggtgacgatggggatcgattaccgtcatggtacgggtaatgaaaatgatctcctttactcaatgcagttccgttatcagtttgataaatcgtggtctcagcaaattgaaccacagtatgttaacgagttaagaacattatcaggcagccgttacgatctggttcagcgtaataacaatattattctggagtacaagaagcaggatattctttctctgaatattccgcatgatattaatggtactgaacacagtacgcagaagattcagttgatcgttaagagcaaatacggtctggatcgtatcgtctgggatgatagtgcattacgcagtcagggcggtcagattcagcatagcggaagccaaagcgcacaagactaccaggctattttgcctgcttatgtgcaaggtggcagcaatatttataaagtgacggctcgcgcctatgaccgtaatggcaatagctctaacaatgtacagcttactattaccgttctgtcgaatggtcaagttgtcgaccaggttggggtaacggactttacggcggataagacttcggctaaagcggataacgccgataccattacttataccgcgacggtgaaaaagaatggggtagctcaggctaatgtccctgtttcatttaatattgtttcaggaactgcaactcttggggcaaatagtgccaaaacggatgctaacggtaaggcaaccgtaacgttgaagtcgagtacgccaggacaggtcgtcgtgtctgctaaaaccgcggagatgacttcagcacttaatgccagtgcggttatattttttgatggtgcgcccgggaagcttgaaaacctgtacttccaaggtacgcgtcaggtgcagctggtggaaaccggcggcggcctggtgcagccgggcggcagcctgcgcctgagctgcgcggcgagcggctttacctttagcagcgtgtatatgaactgggtgcgccaggcgccgggcaaaggcccggaatgggtgagccgcattagcccgaacagcggcaacattggctataccgatagcgtgaaaggccgctttaccattagccgcgataacgcgaaaaacaccctgtatctgcagatgaacaacctgaaaccggaagataccgcgctgtattattgcgcgattggcctgaacctgagcagcagcagcgtgcgcggccagggcacccaggtgaccgtgagcagcggatccgaaaacctgtacttccaaggtgactacaaggacgatgacgataagtggagccatccgcagtttgagaaatctagataaggtaccactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttatacctagggtacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcccgggtgatctgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtccctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatc

SEQ ID NO: 6 - DNA, 5875 bp, Artificial Sequence
  Synthetic plasmid
gacgtcttgacagctagctcagtcctagggattgtgctagcaggtttaatcgaattgacgtctttacggctagctcagtcctaggtactatgctagcaggtttaatcgaattcaaaagatcttttaagaaggagatatacatatgattactcatggttgttatacccggacccggcacaagcataagctaaaaaaaacattgattatgcttagtgctggtttaggattgtttttttatgttaatcagaactcatttgcaaatggtgaaaattattttaaattgggttcggattcaaaactgttaactcatgatagctatcagaatcgccttttttatacgttgaaaactggtgaaactgttgccgatctttctaaatcgcaagatattaatttatcgacgatttggtcgttgaataagcatttatacagttctgaaagcgaaatgatgaaggccgcgcctggtcagcagatcattttgccactcaaaaaacttccctttgaatacagtgcactaccacttttaggttcggcacctcttgttgctgcgggtggtgttgctggtcacacgaataaactgactaaaatgtccccggacgtgaccaaaagcaacatgaccgatgacaaggcattaaattatgcggcacaacaggcggcgagtctcggtagccagcttcagtcgcgatctctgaacggcgattacgcgaaagataccgctcttggtatcgctggtaaccaggcttcgtcacagttgcaggcctggttacaacattatggaacggcagaggttaatctgcaaagtggtaataactttgacggtagttcactggacttcttattaccgttctatgattccgaaaaaatgctggcatttggtcaggtcggagcgcgttacattgactcccgctttacggcaaatttaggtgcgggtcagcgttttttccttcctgcaaacatgttgggctataacgtcttcattgatcaggatttttctggtgataatacccgtttaggtattggtggcgaatactggcgagactatttcaaaagtagcgttaacggctatttccgcatgagcggctggcatgagtcatacaataagaaagactatgatgagcgcccagcaaatggcttcgatatccgttttaatggctatctaccgtcatatccggcattaggcgccaagctgatatatgagcagtattatggtgataatgttgctttgtttaattctgataagctgcaatcgaatcctggtgcggcgaccgttggtgtaaactatactccgattcctctggtgacgatggggatcgattaccgtcatggtacgggtaatgaaaatgatctcctttactcaatgcagttccgttatcagtttgataaatcgtggtctcagcaaattgaaccacagtatgttaacgagttaagaacattatcaggcagccgttacgatctggttcagcgtaataacaatattattctggagtacaagaagcaggatattctttctctgaatattccgcatgatattaatggtactgaacacagtacgcagaagattcagttgatcgttaagagcaaatacggtctggatcgtatcgtctgggatgatagtgcattacgcagtcagggcggtcagattcagcatagcggaagccaaagcgcacaagactaccaggctattttgcctgcttatgtgcaaggtggcagcaatatttataaagtgacggctcgcgcctatgaccgtaatggcaatagctctaacaatgtacagcttactattaccgttctgtcgaatggtcaagttgtcgaccaggttggggtaacggactttacggcggataagacttcggctaaagcggataacgccgataccattacttataccgcgacggtgaaaaagaatggggtagctcaggctaatgtccctgtttcatttaatattgtttcaggaactgcaactcttggggcaaatagtgccaaaacggatgctaacggtaaggcaaccgtaacgttgaagtcgagtacgccaggacaggtcgtcgtgtctgctaaaaccgcggagatgacttcagcacttaatgccagtgcggttatattttttgatggtgcgcccgggaagcttgaaaacctgtattttcagggcacccgtcaggttcagctggttgaaaccggtggtggtctggttcagcctggtggtagcctgcgtctgagctgtgcagcaagcggttttacctttagcagcgtttatatgaattgggttcgtcaggcacctggtaaaggtccggaatgggttagccgtattagcccgaatagcggtaatattggttataccgatagcgtgaaaggtcgctttaccattagccgtgataatgcaaaaaataccctgtacctgcagatgaataatctgaaaccggaagataccgcactgtattattgtgcaattggtctgaatctgagcagcagcagcgttcgtggtcagggtacacaggttaccgtgagcagcggtagcgagaatctgtatttccaaggtgattataaagacgacgatgacaagtggtcccatccgcagtttgaaaaaagccgttaaggtaccactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttatacctagggtacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcccgggtgatctgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtccctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatc

SEQ ID NO: 7 - DNA, 5902 bp, Artificial Sequence
  Synthetic plasmid
gacgtcttgacagctagctcagtcctagggattgtgctagcaggtttaatcgaattgacgtctttacggctagctcagtcctaggtactatgctagcaggtttaatcgaattcaaaagatcttttaagaaggagatatacatatgattactcatggttgttatacccggacccggcacaagcataagctaaaaaaaacattgattatgcttagtgctggtttaggattgtttttttatgttaatcagaactcatttgcaaatggtgaaaattattttaaattgggttcggattcaaaactgttaactcatgatagctatcagaatcgccttttttatacgttgaaaactggtgaaactgttgccgatctttctaaatcgcaagatattaatttatcgacgatttggtcgttgaataagcatttatacagttctgaaagcgaaatgatgaaggccgcgcctggtcagcagatcattttgccactcaaaaaacttccctttgaatacagtgcactaccacttttaggttcggcacctcttgttgctgcgggtggtgttgctggtcacacgaataaactgactaaaatgtccccggacgtgaccaaaagcaacatgaccgatgacaaggcattaaattatgcggcacaacaggcggcgagtctcggtagccagcttcagtcgcgatctctgaacggcgattacgcgaaagataccgctcttggtatcgctggtaaccaggcttcgtcacagttgcaggcctggttacaacattatggaacggcagaggttaatctgcaaagtggtaataactttgacggtagttcactggacttcttattaccgttctatgattccgaaaaaatgctggcatttggtcaggtcggagcgcgttacattgactcccgctttacggcaaatttaggtgcgggtcagcgttttttccttcctgcaaacatgttgggctataacgtcttcattgatcaggatttttctggtgataatacccgtttaggtattggtggcgaatactggcgagactatttcaaaagtagcgttaacggctatttccgcatgagcggctggcatgagtcatacaataagaaagactatgatgagcgcccagcaaatggcttcgatatccgttttaatggctatctaccgtcatatccggcattaggcgccaagctgatatatgagcagtattatggtgataatgttgctttgtttaattctgataagctgcaatcgaatcctggtgcggcgaccgttggtgtaaactatactccgattcctctggtgacgatggggatcgattaccgtcatggtacgggtaatgaaaatgatctcctttactcaatgcagttccgttatcagtttgataaatcgtggtctcagcaaattgaaccacagtatgttaacgagttaagaacattatcaggcagccgttacgatctggttcagcgtaataacaatattattctggagtacaagaagcaggatattctttctctgaatattccgcatgatattaatggtactgaacacagtacgcagaagattcagttgatcgttaagagcaaatacggtctggatcgtatcgtctgggatgatagtgcattacgcagtcagggcggtcagattcagcatagcggaagccaaagcgcacaagactaccaggctattttgcctgcttatgtgcaaggtggcagcaatatttataaagtgacggctcgcgcctatgaccgtaatggcaatagctctaacaatgtacagcttactattaccgttctgtcgaatggtcaagttgtcgaccaggttggggtaacggactttacggcggataagacttcggctaaagcggataacgccgataccattacttataccgcgacggtgaaaaagaatggggtagctcaggctaatgtccctgtttcatttaatattgtttcaggaactgcaactcttggggcaaatagtgccaaaacggatgctaacggtaaggcaaccgtaacgttgaagtcgagtacgccaggacaggtcgtcgtgtctgctaaaaccgcggagatgacttcagcacttaatgccagtgcggttatattttttgatggtgcgcccgggaagcttgaaaacctgtacttccaaggtacgcgtcaggtgcagctgcaggaaagcggtggtggtctggtgcaggccggtggtagcctgcgtctgagctgtgccgccagcggtcgtacctttagcgaatatgccatgggttggtttcgtcaggccccgggtaaagaacgtgaatttgtggccaccattagctggagcggtggtagcacctattataccgatagcgtgaaaggtcgttttaccattagccgtgataatgccaaaaataccgtgtatctgcagatgaatagcctgaaaccggatgataccgccgtgtattattgtgccgccgccggtctgggtaccgtggtgagcgaatgggattatgattatgattattggggtcagggtacccaggtgaccgtgagcagcggtagcggatccgaaaacctgtacttccaaggtgactacaaggacgatgacgataagtggagccatccgcagtttgagaaatctagataaggtaccactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttatacctagggtacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcccgggtgatctgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtccctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatc

SEQ ID NO: 8 - DNA, 4336 bp, Artificial Sequence
  Synthetic plasmid
gacgtcttgacagctagctcagtcctagggattgtgctagcaggtttaatcgaattgacgtctttacggctagctcagtcctaggtactatgctagcaggtttaatcgaattcaaaagatcttttaagaaggagatatacatatgaaagcaaccaagctggttctgggtgccgtgattctgggcagtaccctgttagcaggttgttctagcaatgccaaaatcgaccaaggcatcaacaacaatggcccgacccacgaaaaccagctgggtgccggtgcctttggtggttatcaggtgaacccgtacgtgggctttgaaatgggctatgattggctgggccgcatgccgtacaaaggcagtgtggagaacggcgcctataaagcacagggcgtgcagctgacagcaaaactgggctaccctattaccgacgacctggacatctacacacgcttaggcggcatggtgtggcgcgccgataccaagagcaacgtgtacggcaagaaccacgataccggcgtgagtccggtgtttgccggcggtgtggagtatgcaatcaccccggaaattgccacacgtaagcttgaaaacctgtacttccaaggtacgcgtcaggtgcagctggtggaaaccggcggcggcctggtgcagccgggcggcagcctgcgcctgagctgcgcggcgagcggctttacctttagcagcgtgtatatgaactgggtgcgccaggcgccgggcaaaggcccggaatgggtgagccgcattagcccgaacagcggcaacattggctataccgatagcgtgaaaggccgctttaccattagccgcgataacgcgaaaaacaccctgtatctgcagatgaacaacctgaaaccggaagataccgcgctgtattattgcgcgattggcctgaacctgagcagcagcagcgtgcgcggccagggcacccaggtgaccgtgagcagcggatccgaaaacctgtacttccaaggtgactacaaggacgatgacgataagtggagccatccgcagtttgagaaatctagataaggtaccactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttatacctagggtacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcccgggtgatctgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtccctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatc

SEQ ID NO: 9 - DNA, 4363 bp, Artificial Sequence
  Synthetic plasmid
gacgtcttgacagctagctcagtcctagggattgtgctagcaggtttaatcgaattgacgtctttacggctagctcagtcctaggtactatgctagcaggtttaatcgaattcaaaagatcttttaagaaggagatatacatatgaaagcaaccaagctggttctgggtgccgtgattctgggcagtaccctgttagcaggttgttctagcaatgccaaaatcgaccaaggcatcaacaacaatggcccgacccacgaaaaccagctgggtgccggtgcctttggtggttatcaggtgaacccgtacgtgggctttgaaatgggctatgattggctgggccgcatgccgtacaaaggcagtgtggagaacggcgcctataaagcacagggcgtgcagctgacagcaaaactgggctaccctattaccgacgacctggacatctacacacgcttaggcggcatggtgtggcgcgccgataccaagagcaacgtgtacggcaagaaccacgataccggcgtgagtccggtgtttgccggcggtgtggagtatgcaatcaccccggaaattgccacacgtaagcttgaaaacctgtacttccaaggtacgcgtcaggtgcagctgcaggaaagcggtggtggtctggtgcaggccggtggtagcctgcgtctgagctgtgccgccagcggtcgtacctttagcgaatatgccatgggttggtttcgtcaggccccgggtaaagaacgtgaatttgtggccaccattagctggagcggtggtagcacctattataccgatagcgtgaaaggtcgttttaccattagccgtgataatgccaaaaataccgtgtatctgcagatgaatagcctgaaaccggatgataccgccgtgtattattgtgccgccgccggtctgggtaccgtggtgagcgaatgggattatgattatgattattggggtcagggtacccaggtgaccgtgagcagcggtagcggatccgaaaacctgtacttccaaggtgactacaaggacgatgacgataagtggagccatccgcagtttgagaaatctagataaggtaccactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttatacctagggtacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcccgggtgatctgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtccctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatc

SEQ ID NO: 10 - DNA, 4363 bp, Artificial Sequence
  Synthetic plasmid
gacgtcttgacagctagctcagtcctagggattgtgctagcaggtttaatcgaattgacgtctttacggctagctcagtcctaggtactatgctagcaggtttaatcgaattcaaaagatcttttaagaaggagatatacatatgaaagccaccaaactggttctgggtgcagttattctgggtagcaccctgctggcaggttgtagcagcaatgcaaaaattgatcagggcattaataacaacggtccgacacatgaaaatcagttaggtgccggtgcatttggtggttatcaggttaatccgtatgtgggttttgaaatgggttatgattggctgggtcgtatgccgtataaaggtagcgttgaaaatggtgcatataaagcacagggtgttcagctgaccgcaaaactgggttatccgattaccgatgatctggatatctatacccgtttaggtggtatggtttggcgtgcagataccaaaagcaatgtgtatggcaaaaatcatgataccggtgttagtccggtttttgccggtggtgttgaatatgcaattacaccggaaattgcaacccgcaaactggaaaatctgtattttcagggcacccgtcaggttcagctgcaagaaagcggtggtggtctggttcaggcaggcggtagcctgcgtctgagctgtgcagcaagcggtcgtacctttagcgaatatgccatgggttggtttcgtcaggcaccgggtaaagaacgtgaatttgttgcaaccattagctggtctggtggtagcacctattataccgatagcgttaaaggtcgttttaccattagccgtgataatgccaaaaataccgtttacctgcagatgaatagcctgaaaccggatgataccgcagtgtattattgtgcagcagcaggtctgggtacagttgttagcgagtgggattatgattatgactattggggtcagggtacacaggttaccgttagcagcggtagcggtagtgagaacctgtatttccaaggtgattataaagatgacgatgataagtggtcccatccgcagtttgaaaaaagccgttaaggtaccactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttatacctagggtacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcccgggtgatctgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtccctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatc

SEQ ID NO: 11 - DNA, 5590 bp, Artificial Sequence
  Synthetic plasmid
gacgtcttgacagctagctcagtcctagggattgtgctagcaggtttaatcgaattgacgtctttacggctagctcagtcctaggtactatgctagcaggtttaatcgaattcaaaagatcttttaagaaggagatatacatatgattactcatggttgttatacccggacccggcacaagcataagctaaaaaaaacattgattatgcttagtgctggtttaggattgtttttttatgttaatcagaactcatttgcaaatggtgaaaattattttaaattgggttcggattcaaaactgttaactcatgatagctatcagaatcgccttttttatacgttgaaaactggtgaaactgttgccgatctttctaaatcgcaagatattaatttatcgacgatttggtcgttgaataagcatttatacagttctgaaagcgaaatgatgaaggccgcgcctggtcagcagatcattttgccactcaaaaaacttccctttgaatacagtgcactaccacttttaggttcggcacctcttgttgctgcgggtggtgttgctggtcacacgaataaactgactaaaatgtccccggacgtgaccaaaagcaacatgaccgatgacaaggcattaaattatgcggcacaacaggcggcgagtctcggtagccagcttcagtcgcgatctctgaacggcgattacgcgaaagataccgctcttggtatcgctggtaaccaggcttcgtcacagttgcaggcctggttacaacattatggaacggcagaggttaatctgcaaagtggtaataactttgacggtagttcactggacttcttattaccgttctatgattccgaaaaaatgctggcatttggtcaggtcggagcgcgttacattgactcccgctttacggcaaatttaggtgcgggtcagcgttttttccttcctgcaaacatgttgggctataacgtcttcattgatcaggatttttctggtgataatacccgtttaggtattggtggcgaatactggcgagactatttcaaaagtagcgttaacggctatttccgcatgagcggctggcatgagtcatacaataagaaagactatgatgagcgcccagcaaatggcttcgatatccgttttaatggctatctaccgtcatatccggcattaggcgccaagctgatatatgagcagtattatggtgataatgttgctttgtttaattctgataagctgcaatcgaatcctggtgcggcgaccgttggtgtaaactatactccgattcctctggtgacgatggggatcgattaccgtcatggtacgggtaatgaaaatgatctcctttactcaatgcagttccgttatcagtttgataaatcgtggtctcagcaaattgaaccacagtatgttaacgagttaagaacattatcaggcagccgttacgatctggttcagcgtaataacaatattattctggagtacaagaagcaggatattctttctctgaatattccgcatgatattaatggtactgaacacagtacgcagaagattcagttgatcgttaagagcaaatacggtctggatcgtatcgtctgggatgatagtgcattacgcagtcagggcggtcagattcagcatagcggaagccaaagcgcacaagactaccaggctattttgcctgcttatgtgcaaggtggcagcaatatttataaagtgacggctcgcgcctatgaccgtaatggcaatagctctaacaatgtacagcttactattaccgttctgtcgaatggtcaagttgtcgaccaggttggggtaacggactttacggcggataagacttcggctaaagcggataacgccgataccattacttataccgcgacggtgaaaaagaatggggtagctcaggctaatgtccctgtttcatttaatattgtttcaggaactgcaactcttggggcaaatagtgccaaaacggatgctaacggtaaggcaaccgtaacgttgaagtcgagtacgccaggacaggtcgtcgtgtctgctaaaaccgcggagatgacttcagcacttaatgccagtgcggttatattttttgatggtgcgcccgggaagcttgtcgacggagctcgataatccggcaaaaaagggcaaggtgtcaccaccctgccctttttctttaaaaccgaaaagattacttcgcgttatgcaggcttcctcgctcactgactcgctgcgctcggtcgttcggctgcggcgagcggtatcagctcactcaaaggcggtaatctcgagtcgatccaaactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttatacctagggtacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcccgggtgatctgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtccctagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatc

(19) United States
(12) Patent Application Publication
(10) Pub. No.: US 2025/0026812 A1
(43) Pub. Date: Jan. 23, 2025

(54) Engineered Probiotics for Treatment and Immunity against Viruses

(71) Applicant: University of Cincinnati, Cincinnati, OH (US)

(72) Inventors: Nalinikanth Kotagiri, West Chester, OH (US); Shindu Thomas, Cincinnati, OH (US); Nitin S. KAMBLE, Cincinnati, OH (US)

(73) Assignee: University of Cincinnati, Cincinnati, OH (US)

(21) Appl. No.: 17/919,424

(22) PCT Filed: Apr. 19, 2021
(86) PCT No.: PCT/US21/28027

§ 371 Date: Oct. 17, 2022

Related U.S. Application Data
(60) Provisional application No. 63/011,446, filed on Apr. 17, 2020.

(51) Int. Cl. C07K 16/10; A61K 35/00; A61K 35/741; A61P 31/14

(52) U.S. Cl. CPC C07K 16/1003; A61K 35/741; A61P 31/14; A61K 2035/115; C07K 2317/569

(57) ABSTRACT

The present invention involves an engineered probiotic bacterium comprising a heterologous nucleic acid, where the heterologous nucleic acid comprises a nucleic acid sequence encoding an anti-spike glycoprotein nanobody of a coronavirus. In one embodiment, the bacterium is Escherichia coli Nissle 1917. In another embodiment, the anti-spike glycoprotein nanobody appears on the surface of the probiotic bacteria.

### CROSS REFERENCE TO RELATED APPLICATIONS

This application is a continuation of PCT Application No. PCT/US21/28027 filed Apr. 19, 2021, which claims benefit of U.S. Provisional Application Ser. No. 63/011,446, filed Apr. 17, 2020, which application is hereby incorporated by reference in its entirety.

### SEQUENCE LISTING

A CRF sequence listing in ASCII format, having file name “2024-08-02_Sequence_Listing_ST25.txt” (51,486 bytes), created on Aug. 2, 2024, is incorporated herein by reference in its entirety.

### TECHNICAL FIELD

The present invention relates to a novel engineered probiotic.

### BACKGROUND OF THE INVENTION

The Covid-19 pandemic has revealed that the SARS-Cov-2 has multiple routes of transmission, including the fecal-oral route, as evidenced by persistent diarrhea, and shedding via feces. Structural analysis has revealed that the receptor binding domain (RBD) of the spike glycoprotein on SARS-Cov-2 interacts strongly with angiotensin converting enzyme 2 (ACE2). Besides lung, ACE2 is predominantly expressed in intestines, testis, and kidney. Therefore, strategies involving blocking the interaction of ACE2 with spike proteins might help in combating the virus. While such strategies that involve development of antibodies and small molecules could prove to be immensely beneficial in the short run as therapeutics, a more holistic approach is needed to both treat and provide long-lasting immunity against such viruses, such that recurrence is prevented and new transmissions effectively blocked.

### SUMMARY OF THE INVENTION

The present invention involves an engineered probiotic bacterium comprising a heterologous nucleic acid, where the heterologous nucleic acid comprises a nucleic acid sequence encoding an anti-spike glycoprotein nanobody of a coronavirus. In one embodiment, the bacterium is Escherichia coli Nissle 1917. In another embodiment, the nucleic acid sequence is selected from the group consisting of SEQ ID NO: 1, SEQ ID NO: 2, SEQ ID NO: 3 and SEQ ID NO: 4. In another embodiment, the anti-spike glycoprotein nanobody appears on the surface of the probiotic bacteria.

In one embodiment, the heterologous nucleic acid is located in a plasmid. In another embodiment, the plasmid is selected from the group consisting of pNKLab001, pNKLab002, pNKLab003 and pNKLab004. In one embodiment, the plasmid further incorporates a surface display signal. In another embodiment, the surface display signal is selected from the group consisting of Intimin and Lpp-OmpA. In another embodiment, the plasmid has a sequence selected from the group consisting of SEQ ID NO: 5, SEQ ID NO: 6, SEQ ID NO: 7, SEQ ID NO: 8, SEQ ID NO: 9 and SEQ ID NO: 10.

In another embodiment, the present invention involves a plasmid that expresses one or more surface displayed nanobodies where the one or more surface displayed nanobodies have a sequence selected from the group consisting of SEQ ID NO: 1, SEQ ID NO: 2, SEQ ID NO: 3 and SEQ ID NO: 4.

In one embodiment, the plasmid further incorporates a surface display signal. In another embodiment, the surface display signal is selected from the group consisting of Intimin and Lpp-OmpA. In one embodiment, the plasmid has a nucleic acid sequence selected from the group consisting of SEQ ID NO: 5, SEQ ID NO: 6, SEQ ID NO: 7, SEQ ID NO: 8, SEQ ID NO: 9 and SEQ ID NO: 10. In another embodiment, the plasmid is a CJ23 plasmid. In one embodiment, the plasmid has the nucleic acid sequence of SEQ ID NO: 11.

In one embodiment, the present invention involves a pharmaceutical composition including the engineered probiotic bacterium and a pharmaceutically acceptable excipient. In another embodiment, the pharmaceutical composition is formulated for oral administration to a subject. In one embodiment, the pharmaceutical composition is formulated for rectal administration to a subject. In another embodiment, the pharmaceutical composition is formulated as a pill, a capsule, a lozenge or a suppository.

In one embodiment, the present invention involves a method for preventing a disease or disorder in a subject. The method involves administering the engineered probiotic bacterium of the present invention to the subject, wherein the engineered probiotic bacterium expresses an anti-spike glycoprotein nanobody, thereby preventing the disease or disorder in the subject.

In another embodiment of the method, the engineered probiotic bacterium is administered orally. In one embodiment of the method, the engineered probiotic bacterium is administered rectally.

### BRIEF DESCRIPTION OF THE DRAWINGS

The foregoing summary, as well as the following detailed description of preferred embodiments of the application, will be better understood when read in conjunction with the appended drawings.

FIG. 1 is a schematic representation of an engineered probiotic (EP) with surface expressing nanobodies binding to virus and spike protein antigen and presenting to intestinal dendritic cells. Not drawn to scale.

FIG. 2 is a schematic representation of Human IgG Immunoglobulin, Llama Immunoglobulins and a VHH Single domain antibody.

FIG. 3 is a series of linear maps for four constructs of different combinations of the nanobodies Ty1 and VHH72 fused with surface display signals Intimin and Lpp-OmpA.

FIG. 4A is a circular map of pNKLab001-Intimin-Ty1Nb-Flag/Strep-Tag.

FIG. 4B is a linear map of pNKLab001-Intimin-Ty1Nb-Flag/Strep-Tag.

FIG. 5A is a circular map of pNKLab002-Intimin-VHH72Nb-Flag/Strep-Tag.

FIG. 5B is a linear map of pNKLab002-Intimin-VHH72Nb-Flag/Strep-Tag.

FIG. 6A is a circular map of pNKLab003-Lpp-OmpA-Ty1Nb-Flag/Strep-Tag.

FIG. 6B is a linear map of pNKLab003-Lpp-OmpA-Ty1Nb-Flag/Strep-Tag.

FIG. 7A is a circular map of pNKLab004-Lpp-OmpA-VHH72Nb-Flag/Strep-Tag.

FIG. 7B is a linear map of pNKLab004-Lpp-OmpA-VHH72Nb-Flag/Strep-Tag.

FIG. 8 is an illustration of a nanobody displayed on surface of engineered probiotic.

FIG. 9A is an illustration of a VHH72 nanobody fused to Intimin.

FIG. 9B is an illustration of a Ty1 nanobody fused to Intimin.

FIG. 9C is an illustration of a VHH72 nanobody fused to Lpp-OmpA.

FIG. 9D is an illustration of a Ty1 nanobody fused to Lpp-OmpA.

FIG. 9E is a series of linear maps of the gene blocks for pNKLab001, pNKLab002, pNKLab003 and pNKLab004.

FIG. 10A is an illustration of the structure of FIG. 9A bound to the surface of a bacteria.

FIG. 10B is an illustration of the structure of FIG. 9B bound to the surface of a bacteria.

FIG. 11A is an illustration of the structure of FIG. 9C bound to the surface of a bacteria.

FIG. 11B is an illustration of the structure of FIG. 9D bound to the surface of a bacteria.

FIG. 12 is a schematic of the experimental approach described in Example 12 where the green well represents anticipated positive control.

FIG. 13 is a graph of the experimental results showing the sequestration of spike protein using various embodiments of the present invention.

### DETAILED DESCRIPTION OF THE INVENTION

The details of one or more embodiments of the disclosed subject matter are set forth in this document. Modifications to embodiments described in this document, and other embodiments, will be evident to those of ordinary skill in the art after a study of the information provided herein.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

The term “engineered”, as used herein, refers to a nucleic acid molecule, protein molecule, complex, substance, or entity that has been artificially designed, produced, prepared, synthesized and/or manufactured. Therefore, the engineered product is a non-naturally occurring product.

As used herein, the term “engineered bacterium” or “engineered bacterial cell” refers to a bacterial cell that has been genetically modified from its native state. For instance, an engineered bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Engineered bacterial cells of the disclosure may comprise exogenous nucleotide sequences on plasmids. Alternatively, recombinant bacterial cells may comprise exogenous nucleotide sequences stably incorporated into their chromosome.

As used herein, a “heterologous” gene, “heterologous sequence”, or “heterologous nucleic acid” refers to a nucleic acid sequence that is not normally found in a given cell in nature. As used herein, a heterologous sequence encompasses a nucleic acid sequence that is exogenously introduced into a given cell. “Heterologous gene” includes a native gene, or fragment thereof, that has been introduced into the host cell in a form that is different from the corresponding native gene. A heterologous gene may include a native gene, or fragment thereof, introduced into a non-native host cell. Thus, a heterologous gene may be foreign or native to the recipient cell; a nucleic acid sequence that is naturally found in a given cell but expresses an unnatural amount of the nucleic acid and/or the polypeptide which it encodes; and/or two or more nucleic acid sequences that are not found in the same relationship to each other in nature.

“Probiotic”, as used herein, refers to a live, non-pathogenic microorganism, e.g., a bacterium, which can confer health benefits to a host organism. In some embodiments, the host organism is a mammal. In some embodiments, the host organism is a human. Some species, strains, and/or subtypes of non-pathogenic bacteria are currently recognized as probiotic bacteria. Examples of probiotic bacteria include, but are not limited to, Salmonella typhimurium, Listeria monocytogenes, Staphylococcus epidermidis, Bifidobacterium, Bacteroides, Bacillus, Burkholderia cepacia, Propionibacterium, Fusobacterium, Campylobacter jejuni, Lactobacillus acidophilus, Klebsiella, Bacillus coagulans, Enterococcus and Streptococcus, including Streptococcus oralis. The probiotic may be a variant or a mutant strain of bacterium. Non-pathogenic bacteria may be genetically engineered to enhance or improve desired biological properties, e.g., survivability.

A “pharmaceutical composition,” as used herein, refers to a composition comprising an active ingredient (e.g., a bacterial cell, an inducer, a drug, or a detectable compound) with other components such as a physiologically suitable carrier and/or excipient.

As used herein, the term “pharmaceutically acceptable” or “pharmacologically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Moreover, for animal (e.g., human) administration, it will be understood that compositions should meet sterility, pyrogenicity, general safety and purity standards as required by the FDA Office of Biological Standards.

As used herein, the term “pharmaceutically acceptable excipient” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, disintegrating agents, binders, sweetening agents, flavoring agents, perfuming agents, protease inhibitors, plasticizers, emulsifiers, stabilizing agents, viscosity increasing agents, film forming agents, solubilizing agents, surfactants, preservative and antioxidants can also be present in the formulation. The terms such as “excipient”, “carrier”, “pharmaceutically acceptable excipient” or the like are used interchangeably herein.

As used herein, the term “nanobody” refers to any single variable domain of heavy immunoglobulin chains.

As used herein, the term “plasmid” refers to a construct composed of genetic material (i.e., nucleic acid).

As used herein, the term “surface display signal” refers to a genetic element that is programmed to be displayed on the bacterial cell surface, (e.g. flagella, pili, Intimin or Lpp-OmpA).

As used herein, the term “about,” when referring to a value or to an amount of mass, weight, time, volume, pH, size, concentration or percentage is meant to encompass variations of in some embodiments±20%, in some embodiments±10%, in some embodiments±5%, in some embodiments±1%, in some embodiments±0.5%, and in some embodiments±0.1% from the specified amount, as such variations are appropriate to perform the disclosed method.

While the following terms are believed to be well understood by one of ordinary skill in the art, definitions are set forth to facilitate explanation of the disclosed subject matter. Unless defined otherwise, all technical and scientific terms used herein have the same meaning as commonly understood by one of ordinary skill in the art to which the disclosed subject matter belongs.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The human microbiota is a massive, mostly underexplored niche for short-term immunotherapy and long-term adaptive immunity against viruses. The present invention utilizes a probiotic, commensal bacteria (such as Escherichia coli Nissle 1917 (EcN)) that is already present in cosmic populations inside our gut and genetically-modifies it to give it the power of immunotherapy. Some of the ways by which natural probiotics confer protection to the gastrointestinal environment and immunity from pathogens are through: Direct antagonism, Competitive exclusion, Barrier function and Immune stimulation due their proximity to Dendritic cells (DCs). These intestinal DC, also known as Langerhans cells, are the among the first cells to encounter pathogens/viruses in the gastrointestinal tract and, upon activation, migrate to lymph nodes where they activate and educate T cells to initiate the immune response. Therefore, they are adequately positioned to interact with gut bacteria to absorb, process and present antigens, such as spike glycoproteins from invading virus. DCs are known to be the strongest functional professional antigen presenting cells (APCs), which can absorb, process and present antigens. As the key regulators of innate and adaptive immune responses, DCs are at the center of the immune system and capable of interacting with both B cells and T cells, thereby manipulating the humoral and cellular immune responses. Targeting antigens to DC-specific endocytic receptors has been recently recognized as a promising strategy for designing an effective vaccine that elicits a strong and durable T cell response against different types of pathogens. In fact, it was previously analyzed that DCs have the capacity of to interact with bacteria, and that bacteria can act as “Trojan horses”, delivering heterologous proteins to DCs in a processed form that allows extremely efficient loading of both MHC class I and class II molecules. While commensal bacteria and natural probiotics have some capacity to relay antigens to DC, it is apparent that their capacity is limited because they lack the arsenal to capture and present the antigens effectively, and might not be able to limit the antigens and virus to the extracellular space, without losing viability. Moreover, in cases such as SARS-Cov-2, where lethality is severe, it appears natural defenses are overwhelmed leading to a cascade of reactions manifested in severe morbidity and mortality.

There is a need for new strategies that can provide protection against all subtypes of coronavirus, such as SARS, MERS, Covid-19. The EP platform of the present invention is modular in nature, able to integrate any nanobody as a plug-and-play system, allowing integration of unique nanobodies against distinct viruses and pathogens. The EP has a dual role in construct-therapy for infection by preventing virus and its surface proteins from binding to ACE2, and immune activation through DC antigen presentation and activation. They may be able to also neutralize future emergent coronaviruses before they can cause pandemics. Moreover, the ability of bacteria to proliferate at a rapid rate allows for an amplified response as treatment progresses and is entirely autonomous. As a result, the therapeutic response only increases over time without any external input or additional dosage.

### Engineered Probiotic Bacteria

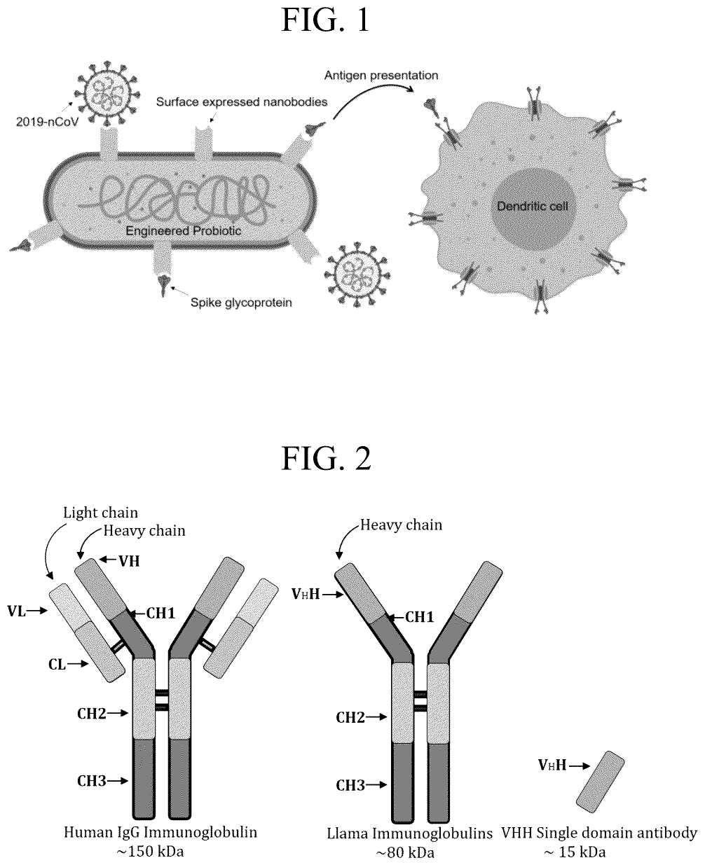

The present invention involves engineered probiotics (EP) capable of synthesizing multiple types of genetically-encoded therapeutic molecules, such as nanobodies. Probiotic bacteria of the present invention are engineered to express unique, coronavirus type-specific nanobodies, and display them on their surface (see FIG. 1) providing them the ability to actively and specifically bind to spike proteins and viruses and exhibit an effect similar to human monoclonal antibodies (mAbs). Besides directly preventing the virus and its antigens from interacting with ACE2 through competitive exclusion and direct antagonism, the EP-nanobody construct will also contribute to immune stimulation by effectively presenting the antigens to DCs for processing, loading and antigen presentation. This is all accomplished without potentially losing its own viability in the process.

An engineered probiotic (EP) with surface nanobodies serves a dual role of neutralizing initial toxicity from virus and its antigens, as well as providing long-term immunity by interfacing with intestinal DC by assisting in antigen absorption and presentation. In one embodiment, E. coli Nissle (EcN) is modified to express and display nanobodies on the surface. This is accomplished using EP that express anti-spike glycoprotein nanobodies on the surface of EcN. Non-limiting examples of other bacteria that can be used for the present invention include Salmonella typhimurium, Listeria monocytogenes, Staphylococcus epidermidis, Bifidobacterium, Bacteroides, Bacillus, Burkholderia cepacia, Propionibacterium, Fusobacterium, Campylobacter jejuni, Lactobacillus acidophilus, Klebsiella, Bacillus coagulans, Enterococcus and Streptococcus, including Streptococcus oralis.

### Plasmids

Surface-bound antigen expression uses the bacterial chassis as an adjuvant to promote immune cell recognition and uptake. The present invention has developed a series of plasmids that constitutively express the surface displayed nanobodies with the help of surface display signals-Intimin and Lpp-OmpA. A plasmid with constitutive promoter CJ2310 accessed from BioBrick, was used as a backbone for the construction of all the pNKLab00 series of plasmids. Two different surface display signals (Intimin/Lpp-OmpA) were evaluated for displaying the COVID-19 nanobodies on the bacterial cell surface. The truncated version of Intimin (Part: BBa_K2332010) and Lpp-OmpA (Part: BBA_J36850) sequences were accessed from the Registry of Standard Biological Parts (iGEM). Intimin is an outer membrane protein and thus proteins fused to its N-termini are known to be displayed on the cell surface. While Lpp-OmpA protein is an outer membrane protein expression system, which consists of 20 amino acid (aa) of signal sequence, the 9 N-terminal amino acids of the lipoprotein (Lpp) and the residual 46-159 aa of the OmpA protein. The Lipoprotein (Lpp) is the most abundant protein on the outer membrane that possesses the function of targeting to the outer membrane, while OmpA domain constitutes 8-stranded, β-barrel to construct an anchor on the outer membrane that provides stable expression of the protein displayed on the outer membrane. By hijacking the efficient targeting OmpA to the outer membrane, it allows C-terminal fusion of the protein sequence to be displayed out of the outer membrane.

The DNA sequences for several plasmids that are useful in the present invention include SEQ ID NO: 5 (pNKLab001_Intimin-TyNb), SEQ ID NO: 6 (variation of pNKLab001-Intimin-TyNb), SEQ ID NO: 7 (pNKLab002_Intimin-VHH72), SEQ ID NO: 8 (pNKLab003_OmpA-TyNb), SEQ ID NO: 9 (pNKLab004_OmpA-VHH72Nb) and SEQ ID NO: 10 (variation of pNKLab004_OmpA-VHH72). In addition, The DNA sequence for a CJ23 plasmid is shown as SEQ ID NO: 11.

### Nanobody

A typical antibody is ˜150 kDa in size which has heavy and light chains covalently joined with Sulphur-Sulphur (S—S) bridges, as shown (FIG. 2). A new class of antibodies found in Llama has only heavy chains bonded with S—S bonds and are ˜80 kDa in size. However, recently single domain antibodies have been discovered called Nanobodies, which are 10 times smaller than a typical antibody that weighs ˜15-18 kDa.

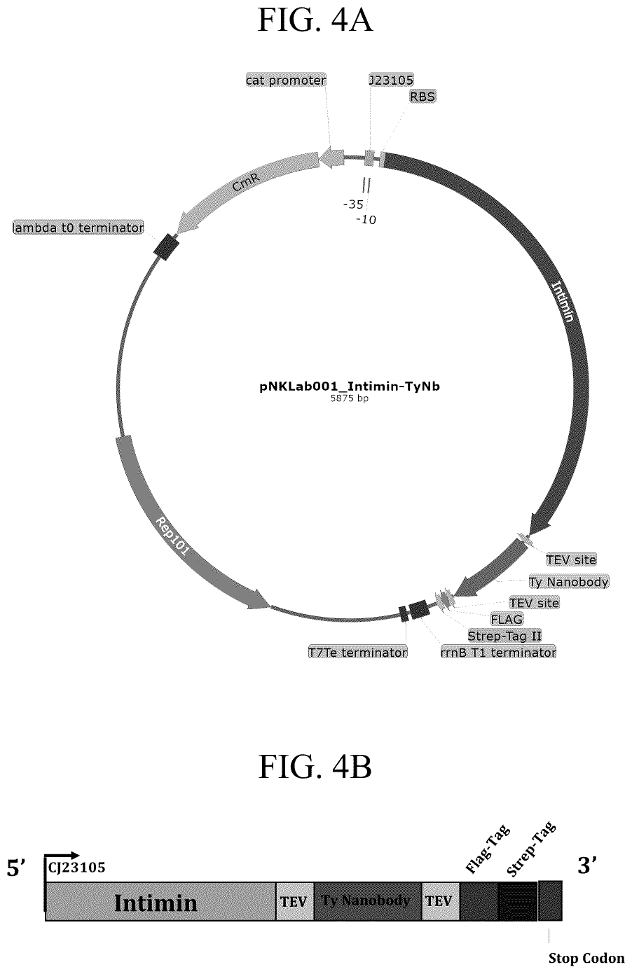

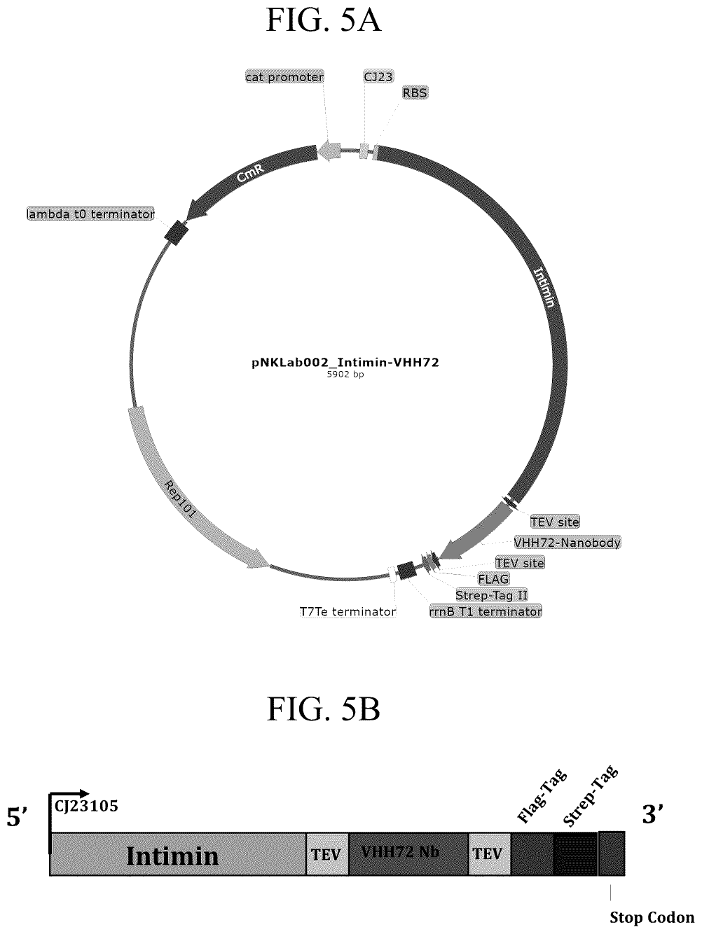

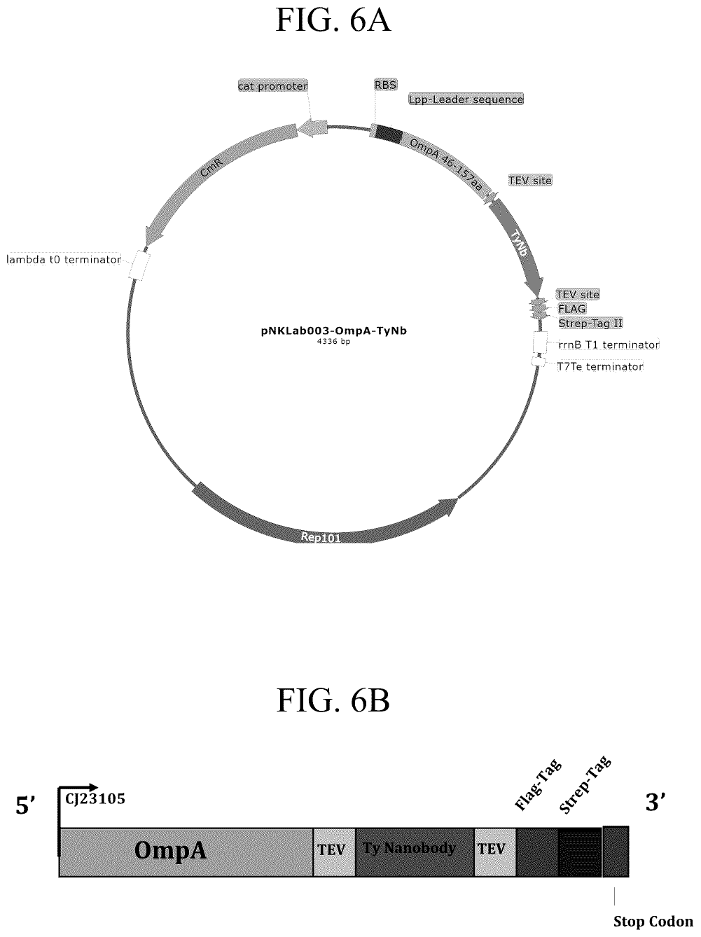

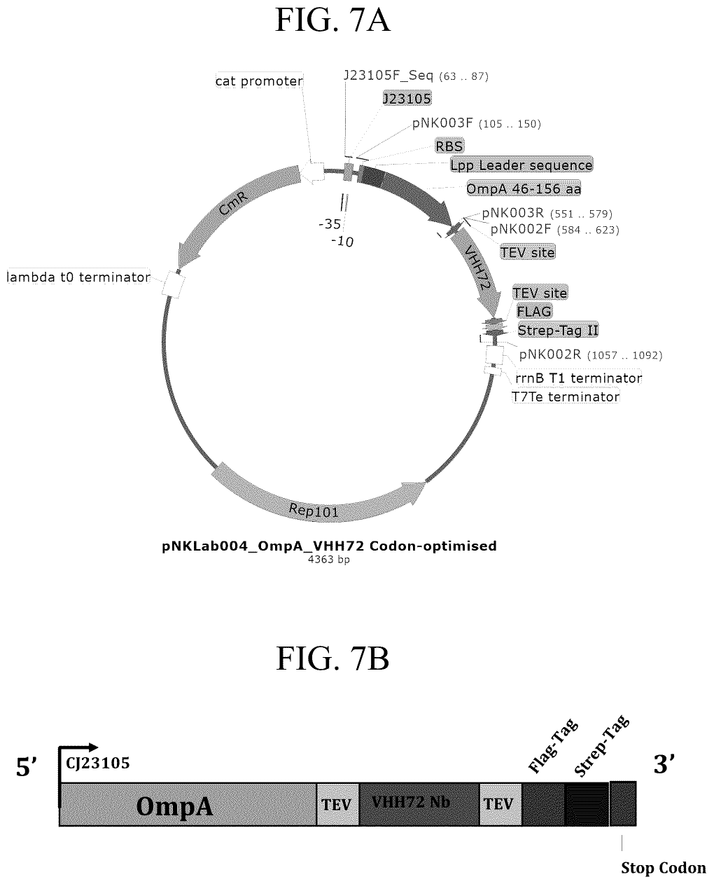

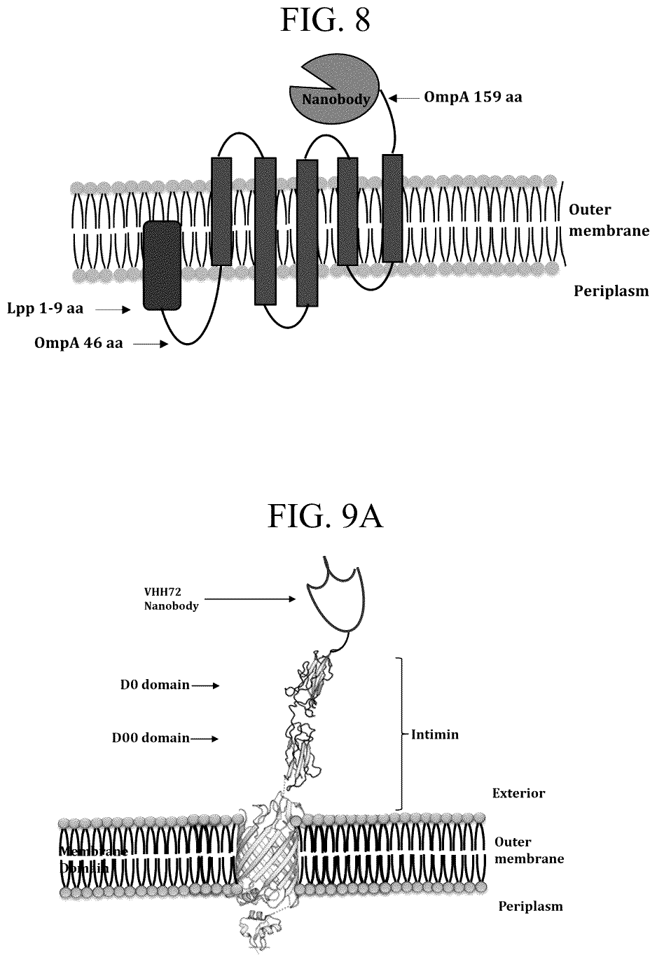

VHH72 and Ty1 nanobody amino acids sequences reported against SARS Cov2 spike protein were accessed. The nanobody sequences were generated de novo utilizing a gene synthesis technology with the flanking BioBrick prefixes/suffixes, codon optimized using Online tools offered by GeneArt Synthesis (Thermo Scientific) and IDT. These gene blocks for Intimin, Lpp-OmpA surface display signals and the COVID19 nanobodies were assembled using Chloramphenicol resistant CJ23 plasmid in order to generate the pNKLab001-pNKLab004 constructs shown in FIGS. 4A, 5A, 6A and 7A.

The DNA sequences for several nanobodies that are useful in the present invention include SEQ ID NO: 1 (VHH72 Nanobody), SEQ ID NO: 2 (variation of a VHH72 Nanobody), SEQ ID NO: 3 (Ty Nanobody) and SEQ ID NO: 4 (variation of a Ty Nanobody).

The engineered probiotic bacteria with surface displayed nanobodies of the present invention serve the dual function of neutralizing initial events in toxicity due to viruses and its antigen, and also provide long-term immunity by means of interacting with intestinal DC by assisting in the antigen absorption and presentation.

As shown in the examples below, the present invention has shown successful expression of the both Ty1 and VHH72 nanobody using both Lpp-Omp and Intimin surface display signals. In addition, pNKLab003 has higher expression of nanobody (Ty1) not only in cell fraction but also in supernatant. Hence, the data shows that the present invention has successfully expressed both the COVID-19 nanobodies on the surface of E. coli Nissle.

### EXAMPLES

### Example 1

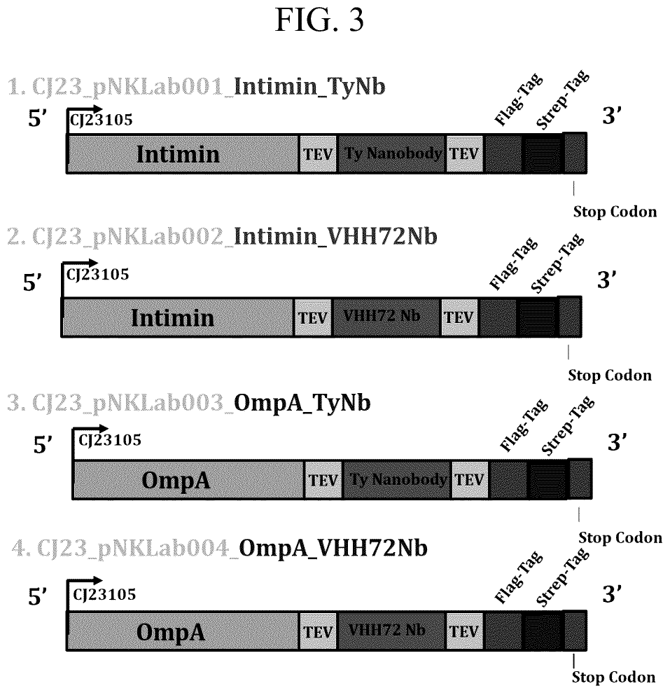

The design, construction and cloning of pNKLab001-4 plasmids for nanobody expression using surface display signals Intimin and Lpp-OmpA in E. coli Nissle is described. For the molecular cloning of COVID Nanobodies VHH72 and Ty1, Chloramphenicol resistant CJ23 plasmid was utilized, as it contains a constitutive promoter CJ23105. Both the Nanobodies, Ty1 and VHH72 were fused with surface display signals Intimin and Lpp-OmpA in order to generate four constructs (See FIG. 3). The Intimin gene sequence is about 2.0 kb corresponding to ˜72 kDa peptide. In order to test and avoid the possibility of large peptide burying the smaller nanobody (13-15 kDa), a shorter surface display signal-Lpp-OmpA of 478 bp, corresponding to ˜15.394 kDa peptide was used. All the constructs were incorporated with Flag tag and Strep tag for the detection and purification purposes. Finally, Tobacco Etch virus (TEV) cleavage sites were incorporated in-between surface display signal and the nanobody for detaching the display signal peptide from that of the nanobody.

### Example 2

For the construction of pNKLab001-Intimin-Ty1Nb Nanobody, a gene block was ordered from a GeneArtSynthesis (Thermo Scientific). It was received lyophilized in 5 μg Kanamycin resistant plasmid pMK-RQ. Upon arrival, it was re-suspended in 50 μL of 1×TAE buffer and chemically transformed into DH5α competent cells (NEB) and spread on LB-kanamycin plates. Following 24 hr incubation at 37° C., positive clones were selected and grown in 3 ml cultures overnight, in order to extract the pKM-RQ plasmid. pKM-RQ and CJ23 plasmids were digested overnight using EcoRI and PspXI and BglII-PspXI restriction enzymes.

The insert released following restrict digest of pKA-RQ was cloned into similarly digested CJ23 plasmid and were ligated overnight at 16° C. 3.5 μL of ligation mixtures was chemically transformed into DH5α competent cells (NEB) and spread on chloramphenicol selection plates. Plates were incubated at 37° C. overnight. About 40-colonies were randomly screened for successful cloning by using colony PCR.

Following Colony PCR, 5 colonies were selected and grew overnight in 3 ml using Chloramphenicol at 37° C. Plasmids were extracted from these 5-colonies using CloneJet plasmid extraction kit (Thermo Scientific) and sent for Sanger sequencing. Sequencing results were verified for mutations manually or by using software tools such as SnapGene and/or Multalign.

### Example 3

High Fidelity PCR amplification for VHH72 Nanobody is described. For the construction of pNKLab002-Intimin-VHH72 Nanobody, the VHH72-Nanobody fused with Flag/Strep tag was amplified by using pNKLab004 plasmid as a template. pNKLab004 plasmid contains, sequentially Lpp-OmpA-VHH72-Flag/Strep. pNKLab004-Lpp-OmpA-VHH72 was constructed prior to this pNKLab002 construct, for which gene block was ordered, as previously, from GeneArtSynthesis (Thermo Scientific).

Amplified PCR product for VHH72-strep/Flag was digested along with CJ23 plasmid by using HindIII- and PspXI restriction enzymes, for placing the VHH72-Flag/strep gene sequence downstream of Intimin in the CJ23 plasmid. This produced the newly constructed plasmid named-pNKLab002-Intimin-VHH72Nb-Flag/strep. Following restrict digest, digest reactions were run on 1% Agarose gel. These were gel extracted, cleaned and ligated overnight.

Following overnight Ligation, 3.5 μL of ligation mixtures was chemically transformed into DH5α competent cells (NEB) and spread on chloramphenicol selection plates. Plates were incubated at 37° C. overnight. About 40-colonies were randomly screened for successful cloning by performing colony PCR on the selected colonies.

### Example 4

Preliminary confirmation with Colony PCR. Following colony PCR, 6 colonies were selected and grew overnight in 3 ml using Chloramphenicol at 37° C. Plasmids were extracted from these 6-colonies using CloneJet plasmid extraction kit (Thermo Scientific) and sent for Sanger sequencing. Sequencing results were verified for mutations manually or by using software tools such as SnapGene and/or Multalign and NCBI.

### Example 5

In order to construct a pNKLab003-Lpp-OmpA-TyNb-Flag/Strep-Tag plasmid, previously constructed pNKLab001-Intimin-Ty1Nb-Flag/Strep-Tag plasmid was used as it contains the Ty1 Nanobody attached with Intimin. The Intimin was replaced by Lpp-OmpA signal, resulting in a pNKLab003-Lpp-OmpA-TyNb-Flag/Strep-Tag plasmid. A Lpp-OmpA signal of about ˜497 bp was PCR amplified by using pNKLab004-Lpp-OmpA-VHH72Nb-Flag/Strep-Tag as a template for PCR amplifications (since it contains the Lpp-OmpA signal). PCR was run on 1% agarose gel.

### Example 6

High Fidelity PCR was used to amplify the Lpp-OmpA signal for cloning to pNKLab001 plasmid. The PCR was cleaned and digested along with pNKLab001-Intimin-Ty1Nb-Flag/Strep-Tag plasmid, by using EcoRI-HindIII restriction enzymes. Following restrict digest with these enzymes, pNKLab001 releases Intimin. Restriction digest of pNKLab001 and Lpp-OmpA signal was done with EcoRI-HindIII. The remaining backbone of pNKLab001 plasmid following the release of Intimin still contains the Ty1 nanobody. The top band of pNKLab001 and digested PCR product for Lpp-OmpA signal were ligated overnight as previously described. Following overnight ligation, 3.5 μL of ligation mixtures was chemically transformed into DH5α competent cells (NEB) and spread on chloramphenicol selection plates. Plates were incubated at 37° C. overnight. About 40-colonies were randomly screened for successful cloning by performing colony PCR on the selected colonies.

### Example 7

Preliminary confirmation was done with Colony PCR for pNKLab003_(OmpA-Ty1Nb-Strp/Flag Tag). Following colony PCR confirmation, 5 colonies were selected and grown overnight in 3 ml using Chloramphenicol at 37° C. Plasmids were extracted from these 5-colonies using CloneJet plasmid extraction kit (Thermo Scientific) and sent for Sanger sequencing. Sequencing results were verified for mutations manually or by using software tools such as SnapGene and/or Multalign.

### Example 8

Confirmation of nanobody expression was conducted with SDS-PAGE and western immunoanalysis. Following the final confirmation of successful molecular cloning of the Nanobodies and surface display signals, these constructs were ready for testing the expression of Nanobodies. Therefore, these constructs were chemically transformed into E. coli Nissle (EcN) and were spread on respective antibiotic selection plates. Plates were incubated at 37° C. overnight. After 24 hrs, a single colony each was picked from these plates for ALL the 4-nanobody expression constructs, pNKLab001, 002, 003 and 004 and grown in LB broth supplemented with chloramphenicol and incubated at 37° C. overnight. 1% of these cultures were inoculated into fresh 10 ml LB broth supplemented with chloramphenicol and grown until OD₆₀₀ reaches 0.9-1.0 (approx. 3.00 hr). These cultures were then centrifuged at 4° C. for 15 min at 3500×g and cells were resuspended in 2 ml of the media. These cells were redistributed in 500 μL 1.5 ml Eppendorf tube.

Both the tubes containing resuspended cells were centrifuged at 4° C. for 15 min at 13,000×g. For the 500 μL cells containing eppendorf's, supernatant was discarded and labelled as cell fraction. In the case of the Eppendorf's that contained 1.5 ml re-suspended cells, cell pellet was discarded and 1350 μL supernatant was collected in another Eppendorf and placed on ice. To these Eppendorf's, 150 μL of ice-cold Tri-Chloro Acetic Acid (TCA) was added, incubated on ice for 30 min, following which these were centrifuged at 4° C. for 15 min at 3500×g. The supernatant was discarded, and the pellet was washed with 950 μL of ice-cold Acetone and centrifuged at 4° C. for 15 min at 3500×g. Following centrifuge, the entire contents of the tube was decanted and air dried, prior to adding 2×SDS-loading buffer. The cell fraction and supernatant containing tubes were added with 2× loading buffer according to their OD₆₀₀ by using the following equation: a) Loading Buffer for cell pellet: OD₆₀₀×200/2; b) Loading buffer for cell supernatant: OD₆₀₀×1500×50. Both of these cell fraction and cell supernatants were then heated at 95° C. for 15 min using a heat block, with intermittent shaking.

Meanwhile, 15% SDS-PAGE gels were prepared and placed in a SDS-running gel tank (BioRad) containing 1×SDS running buffer. The cell Fraction and cell supernatants were loaded on the gel and run at 95° C. for 2.5 3.0 hr. The gels were then transformed to Nitrocellulose paper by using Trans-blot Turbo transfer system (BioRad). Blots were then blocked using 5% milk in 0.1%, 1×TBS Tween buffer overnight at 4° C. These were then washed 3× using 0.1%, 1×TBS Tween and added with 1×TBS buffer. It was added with 1:500 HRP-conjugated Flag-Tag Antibody and incubated for 4.00 hr at room temperature.

These blots were then washed again 3× times with 0.1%, 1×TBW Tween buffer, and transferred into a suitable box and added with equal volumes of Chemiluminescent Dark and Light substrates (Super Signal, West Pico Plus, Thermo Scientific) and incubated for 5 min in dark at RT. These blots were then developed using ChemiDoc Imager (BioRad). The gels were subjected to Silver staining using Pierce™ Silver staining kit (Thermo Scientific), according to manufacturer's protocol.

The Covid-19 nanobodies were expressed on the bacterial cell surface using both Intimin and Lpp-OmpA secretion signals. The Nanobody constructs we generated viz. Intimin-VHH72, Intimin-Ty1, Lpp-OmpA-VHH72 and Lpp-OmpA-Ty1 were expressed in E. coli Nissle. SDS-PAGE Western blot analysis was performed for the confirmation of the nanobody expression using HRP conjugated Anti-Flag Tag antibodies (Sigma Aldrich). The nanobody expression was tested intracellularly and in the supernatant by using TCA-Acetone precipitation method. Expression of Ty1 (pNKLab003) and VHH72 (pNKLab004) nanobodies was observed, in cell fraction, which were fused with Lpp-OmpA surface display signal. Expression of Ty1 (pNKLab001) and VHH72 (pNKLab002) nanobodies were also observed in the supernatant.

### Example 9-COVID19 Bio-Assay

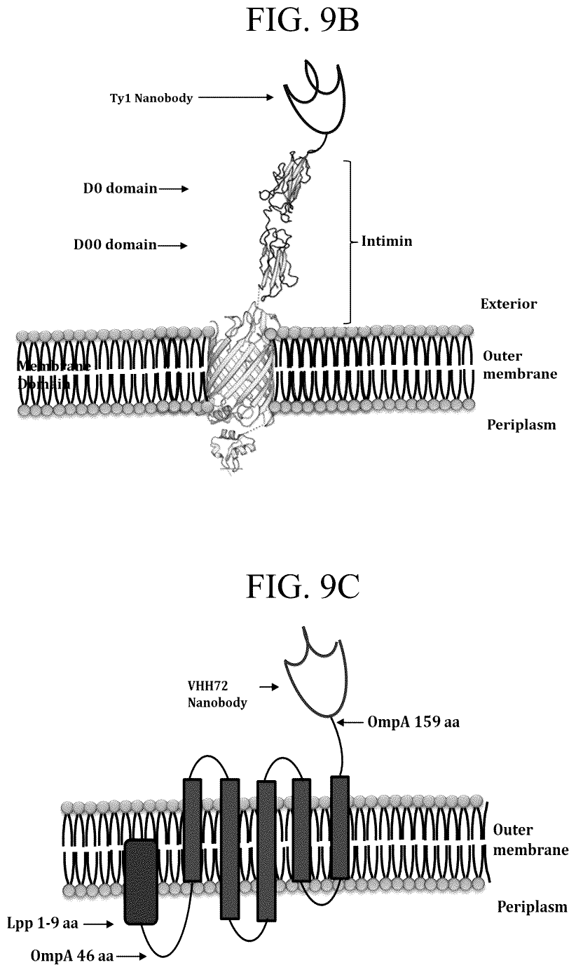

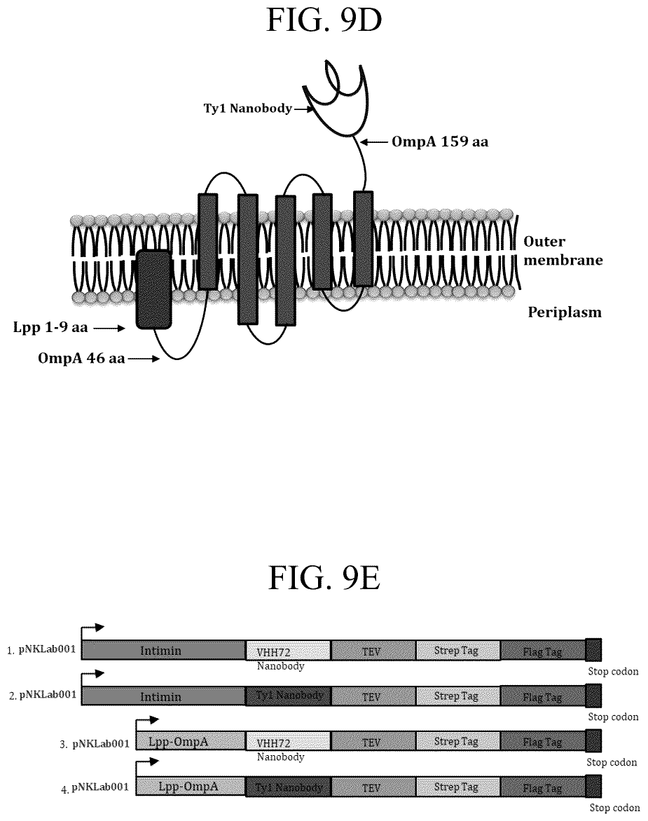

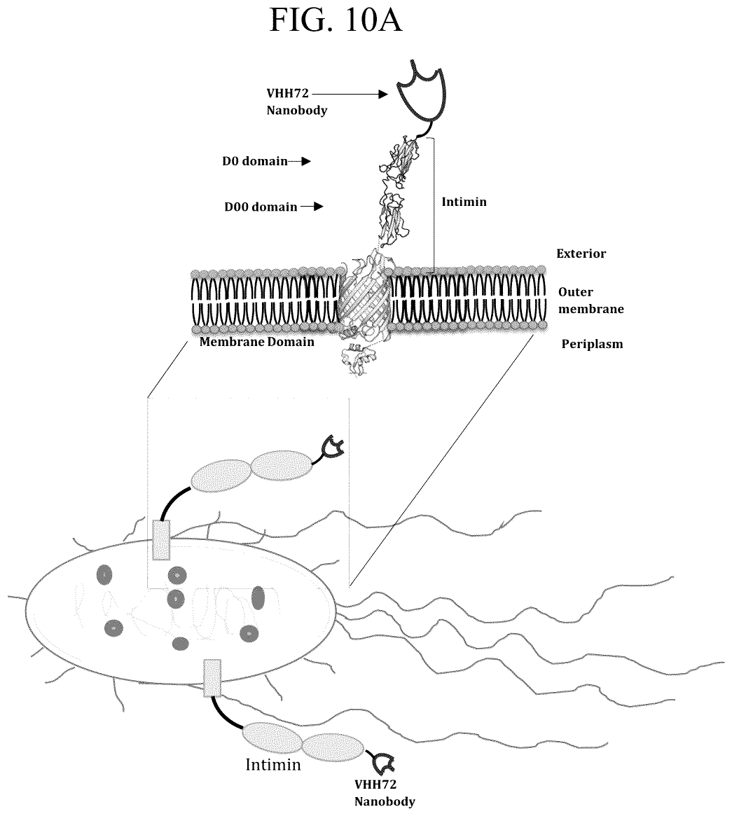

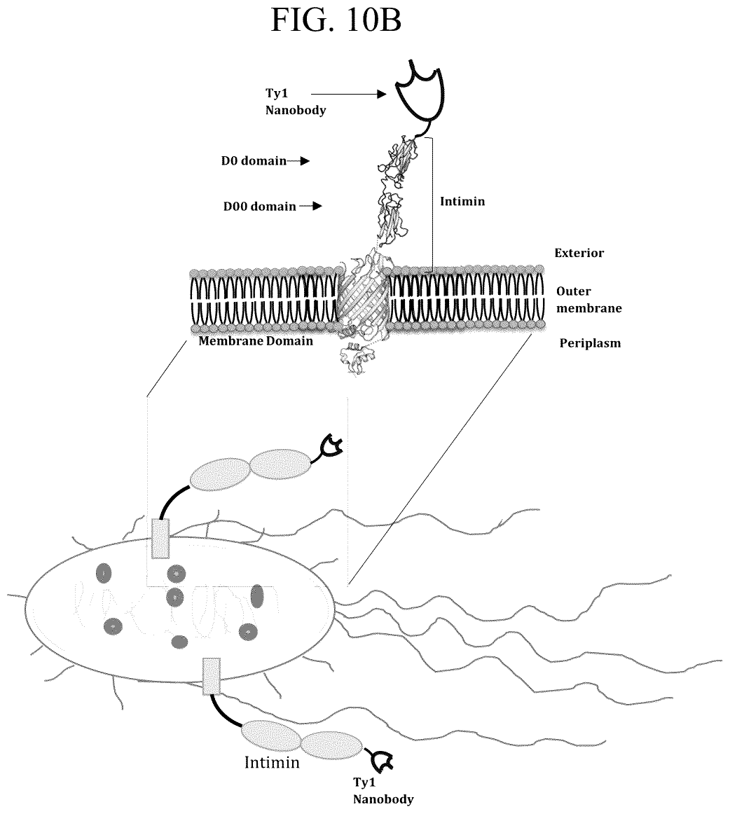

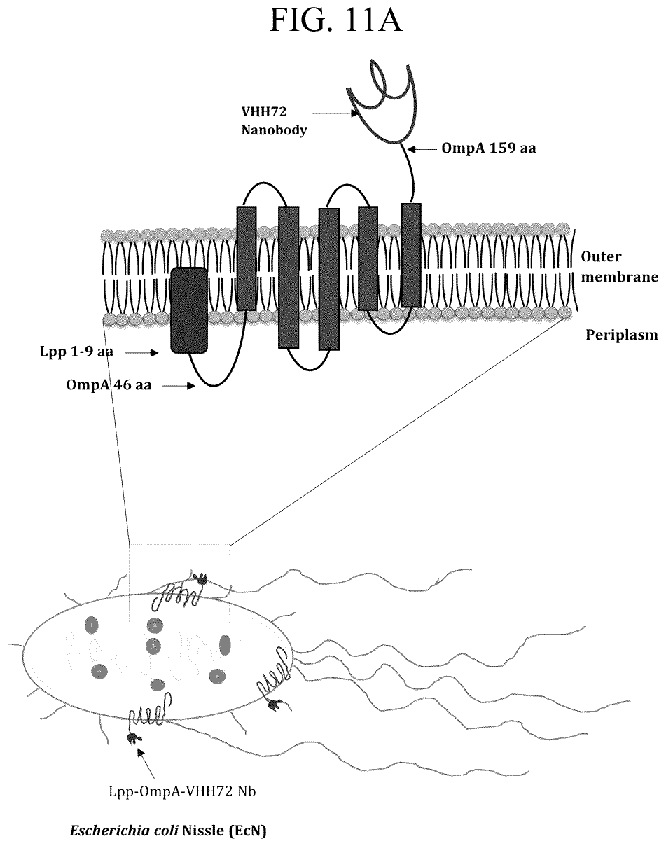

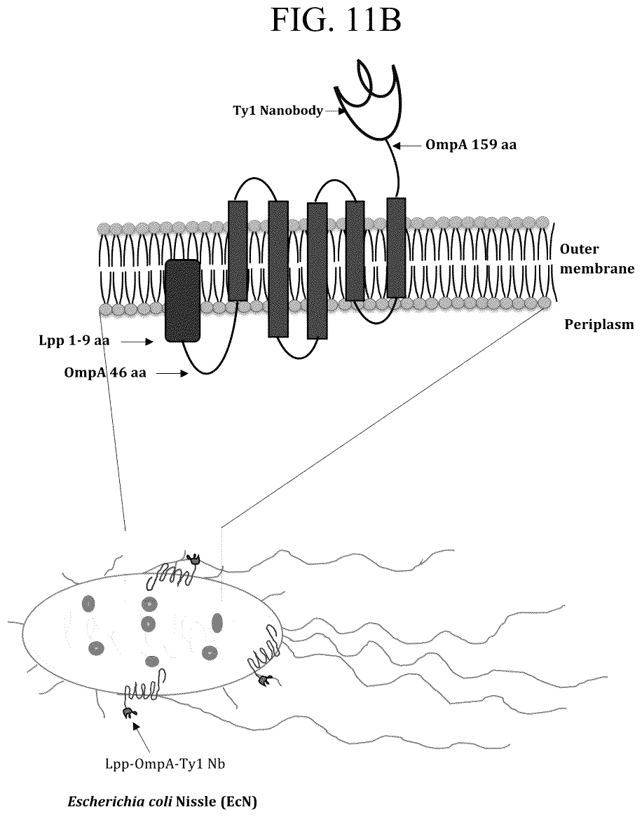

VHH72 and Ty1 nanobody amino acids sequences that were reported against SARS Cov2 spike protein were accessed. The nanobody sequences were generated de novo utilizing a gene synthesis technology with the flanking BioBrick prefixes/suffixes, codon optimized using Online tools offered by GeneArt Synthesis (Thermo Scientific) and IDT. These gene blocks for Intimin, Lpp-OmpA surface display signals and the COVID19 nanobodies were assembled using Chloramphenicol resistant CJ23 plasmid in order to generate the pNKLab001-pNKLab004 constructs shown in FIG. 9E.

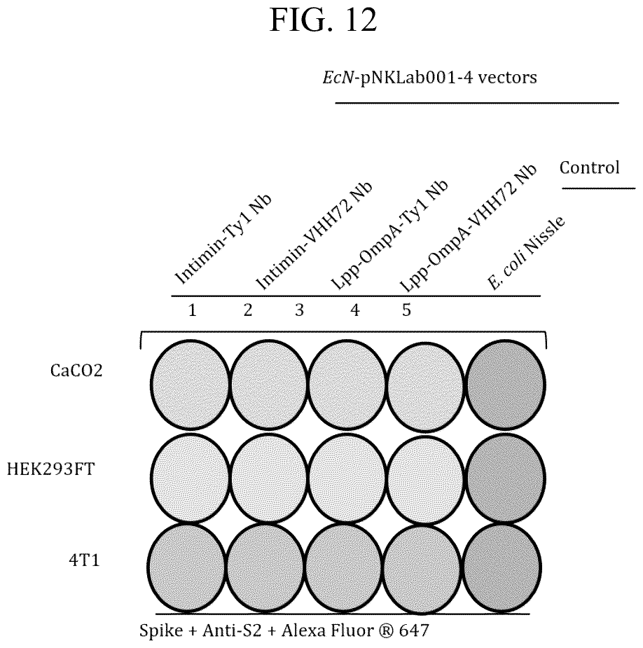

An in-house assay was developed for the determination of the functionality of the nanobody expression on the bacterial cell surface. As the nanobodies-VHH72 and Ty1 bind to the receptor binding domain (RBD) of the SARS-Cov2 spike protein, recombinant SARS-Cov2 S (S1+S2) protein was acquired, where nanobodies would bind to the S1 domain of the spike protein. In order to confirm this binding, the SARS Cov-2 S protein S2 antibody (BioLegend) that specifically binds to the S2 domain of the recombinant spike protein was used. Finally, this whole sandwich complex was visualized by using an AlexaFluor® conjugated anti-mouse IgG2b antibody (BioLegend), using a plate reader-based assay (schematic depicted in FIGS. 12A-12D).

### Example 10

Escherichia coli Nissle were freshly transformed with pNKLab001, pNKLab002, pNKLab003 and pNKLab004 plasmids, while wild-type E. coli Nissle (EcN) was used as a control. All the cultures were grown identically in 10 ml LB medium supplemented with appropriate antibiotic, for approximately 3.00 hr or until OD₆₀₀ reaches 0.8-1.0. All the centrifugation steps were carried out at 3500×g for 15 min, at room temperature (RT) unless otherwise stated.

Following OD₆₀₀ measurements, 1 ml samples from each culture were centrifuged and pellets were resuspended in 1 ml of Phosphate Buffered Saline (PBS). It was centrifuged and pellets were re-suspended in 200 μl of PBS and it was added with 1 μg of Spike Protein (carrier-free Recombinant SARS-COV-2 S Protein (S1+S2) (Bioline #793706)) and incubated in the dark for an hour at RT. Following centrifugation, it was then resuspended in 1 ml of PBS and centrifuged again, and pellet were resuspended into 200 μl of PBS. It was then added with 1 μg of Purified anti-SARS-COV-2 Protein S2 antibody (Bioline #943202), that specifically binds to S2 fragment of spike protein. The whole reaction was incubated in the dark for 30 min. It was centrifuged and resuspended into 1 ml of PBS as previously and centrifuged. Following this, pellet was re-suspended into 200 μl of PBS and added with secondary antibody (Alexa Fluor® 647 anti-mouse IgG2b, Bioline #406715). Following 30 min of incubation in dark, the reactions tubes were centrifuged twice, and pellet were resuspended into 200 μl of PBS. The whole reaction mixture was placed into a transparent 96-well plate and read for fluorescence using a Fluorescent microscope (Leica Microsystems).

### Example 11

In order to identify ACE-2 receptor and evaluate the interaction between ACE-2 receptor and Spike protein, an in-house assay was developed. In addition, a competitive exclusion assay was performed between engineered COVID19 nanobodies (Ty1 and VHH72) and the ACE-2 receptor for binding recombinant spike protein (carrier-free Recombinant SARS-COV-2 S Protein (S1+S2) (Bioline #793706)).

Following cell lines were used for the development of the assay—CaCo2, which is positive for ACE2 receptor, and 4T1 cells, which is negative for ACE2. These cell lines were grown in respective media (ATCC) using an 8-chamber slide (Ibidi), until confluency by incubating at 37° C. with 5% CO2. The cell media were removed, and cell were washed 3 times with Phosphate Buffer Saline (PBS). Cells were then fixed using 4% formaldehyde by incubating for 10 min at room temperature (RT). The formaldehyde was drained, and cells were washed 3× times to remove the residual formaldehyde. It was followed by permeabilization. Cells were permeabilized with permeabilization buffer (0.1% Triton x100 in PBS) and incubated for 10 min at RT, followed by 3 washes with PBS. Cells were then blocked using blocking buffer (3% BSA in PBS with 0.1% Triton X100) for 30 min at RT. Following buffer removal, cells were added with primary antibody and/or spike protein prepared in 0.1% Triton X100, with 30 mg/ml of BSA (Filter sterilized). Primary antibody (Anti-ACE2 (E-11): sc-390851 (Santa Cruz Biotechnology Inc.)) was added for 1-2 hr at RT followed with 3× washes with wash Buffer (0.1% Triton X100 in PBS) (15 min washing with 5 min in between). It was followed with secondary antibody (Alexa Fluor® 647 anti-mouse IgG2b, Bioline #406715) prepared in PBS (0.1% Triton x100 with 30 mg/ml BSA).

When Spike protein was utilized, following the blocking step, and/or Anti-ACE2 washing step, cells were added with 1 μg of Spike Protein (carrier-free Recombinant SARS-CoV-2 S Protein (S1+S2) (Bioline #793706)) and incubated in dark for an hour at RT. Following 3× washes with Washing buffer, 1 μg of Purified anti-SARS-COV-2 Protein S2 antibody (Bioline #943202) was added, that specifically binds to S2 fragment of Spike Protein. The whole reaction was incubated in dark for 30 min. Following 3× washes with washing buffer, secondary antibody (Alexa Fluor® 647 anti-mouse IgG2b, Bioline #406715) was added. After 30 min of incubation in dark, cells were washed 3× times with washing buffer and were visualized for fluorescence, using a fluorescent microscope.

### Example 12

A competitive exclusion assay was used to evaluate sequestration of spike protein. In order to evaluate the competition between engineered COVID19 nanobodies located on bacterial cell surfaces with Intimin or Lpp-OmpA anchor (FIG. 9E) and the ACE-2 receptor located on Cell lines (CACo2, HE293FT and 4T1), for COVID19 Spike protein, an assay was performed (schematic outlined in FIG. 12). The assay was performed as outlined earlier. In brief, the following cell lines were grown until confluency as previously described. In parallel, E. coli Nissle harboring respective surface anchor and Nanobody from an overnight culture was added on the top of the cell lines in the 8-well chamber slides. It was then sequentially added with Spike protein, anti-S2 antibody and AlexaFluor® Antibody as previously described, while wild type E. coli Nissle was used as a positive control.

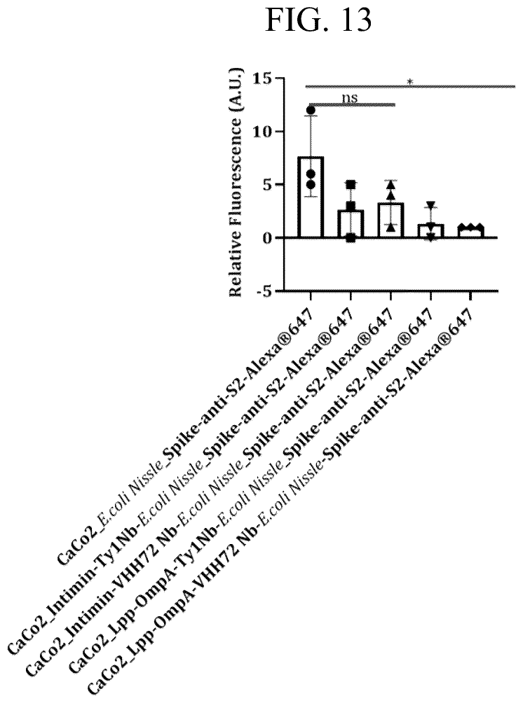

Following fluorescent microscopy, as shown in FIG. 13, spike protein binds to the ACE-2 receptor located on CaCo2 Cell lines enabling the sequential binding of anti-S2 antibody and AlexaFluor Antibody, which is significantly different (p 0.05) than the E. coli Nissle harboring COVID19 nanobodies on the cell surface. It also suggests that when nanobodies are attached with Lpp-OmpA surface display signal, it more efficiently sequesters the spike protein away from the ACE2 receptor than when these are anchored with Intimin (see FIG. 13).

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

### Sequences

SEQ ID NO:1, SEQ ID NO: 2, SEQ ID NO:3 and SEQ ID NO:4 identify the DNA sequences for embodiments of the nanobodies of the present invention.

SEQ ID NO: 5, SEQ ID NO: 6, SEQ ID NO: 7, SEQ ID NO: 8, SEQ ID NO: 9 and SEQ ID NO: 10 identify the DNA sequences for embodiments of the plasmids of the present invention.

SEQ ID NO: 11 identifies the DNA sequence for a CJ23 plasmid.

| SEQ ID NO: 1 | |
|---|---|
| caggtgcagc tgcaggaaag cggtggtggt ctggtgcagg ccggtggtag cctgcgtctg | 60 |
| agctgtgccg ccagcggtcg tacctttagc gaatatgcca tgggttggtt tcgtcaggcc | 120 |
| ccgggtaaag aacgtgaatt tgtggccacc attagctgga gcggtggtag cacctattat | 180 |
| accgatagcg tgaaaggtcg ttttaccatt agccgtgata atgccaaaaa taccgtgtat | 240 |
| ctgcagatga atagcctgaa accggatgat accgccgtgt attattgtgc cgccgccggt | 300 |
| ctgggtaccg tggtgagcga atgggattat gattatgatt attggggtca gggtacccag | 360 |
| gtgaccgtga gcagcggtag c | 381 |
| SEQ ID NO: 2 | |
| aagcttcatc accatcacca tcacggtggt ggtggtagcc aggtgcagct gcaggaaagc | 60 |
| ggtggtggtc tggtgcaggc cggtggtagc ctgcgtctga gctgtgccgc cagcggtcgt | 120 |
| acctttagcg aatatgccat gggttggttt cgtcaggccc cgggtaaaga acgtgaattt | 180 |
| gtggccacca ttagctggag cggtggtagc acctattata ccgatagcgt gaaaggtcgt | 240 |
| tttaccatta gccgtgataa tgccaaaaat accgtgtatc tgcagatgaa tagcctgaaa | 300 |
| ccggatgata ccgccgtgta ttattgtgcc gccgccggtc tgggtaccgt ggtgagcgaa | 360 |
| tgggattatg attatgatta ttggggtcag ggtacccagg tgaccgtgag cagcggtagc | 420 |
| taaaagctt | 429 |
| SEQ ID NO: 3 | |
| caggtgcagc tggtggaaac cggcggcggc ctggtgcagc cgggcggcag cctgcgcctg | 60 |
| agctgcgcgg cgagcggctt tacctttagc agcgtgtata tgaactgggt gcgccaggcg | 120 |
| ccgggcaaag gcccggaatg ggtgagccgc attagcccga acagcggcaa cattggctat | 180 |
| accgatagcg tgaaaggccg ctttaccatt agccgcgata acgcgaaaaa caccctgtat | 240 |
| ctgcagatga acaacctgaa accggaagat accgcgctgt attattgcgc gattggcctg | 300 |
| aacctgagca gcagcagcgt gcgcggccag ggcacccagg tgaccgtgag cagc | 354 |
| SEQ ID NO: 4 | |
| aagcttgaaa acctgtactt ccaaggtgaa ttccaggtgc agctggtgga aaccggcggc | 60 |
| ggcctggtgc agccgggcgg cagcctgcgc ctgagctgcg cggcgagcgg ctttaccttt | 120 |
| agcagcgtgt atatgaactg ggtgcgccag gcgccgggca aaggcccgga atgggtgagc | 180 |
| cgcattagcc cgaacagcgg caacattggc tataccgata gcgtgaaagg ccgctttacc | 240 |
| attagccgcg ataacgcgaa aaacaccctg tatctgcaga tgaacaacct gaaaccggaa | 300 |
| gataccgcgc tgtattattg cgcgattggc ctgaacctga gcagcagcag cgtgcgcggc | 360 |
| cagggcaccc aggtgaccgt gagcagcgga tccgaaaacc tgtacttcca aggtgactac | 420 |
| aaggacgatg acgataagtg gagccatccg cagtttgaga aatctagaaa gctttaacat | 480 |
| atgactcgag t | 491 |
| SEQ ID NO: 5 | |
| gacgtcttga cagctagctc agtcctaggg attgtgctag caggtttaat cgaattgacg | 60 |
| tctttacggc tagctcagtc ctaggtacta tgctagcagg tttaatcgaa ttcaaaagat | 120 |
| cttttaagaa ggagatatac atatgattac tcatggttgt tatacccgga cccggcacaa | 180 |
| gcataagcta aaaaaaacat tgattatgct tagtgctggt ttaggattgt ttttttatgt | 240 |
| taatcagaac tcatttgcaa atggtgaaaa ttattttaaa ttgggttcgg attcaaaact | 300 |
| gttaactcat gatagctatc agaatcgcct tttttatacg ttgaaaactg gtgaaactgt | 360 |
| tgccgatctt tctaaatcgc aagatattaa tttatcgacg atttggtcgt tgaataagca | 420 |
| tttatacagt tctgaaagcg aaatgatgaa ggccgcgcct ggtcagcaga tcattttgcc | 480 |
| actcaaaaaa cttccctttg aatacagtgc actaccactt ttaggttcgg cacctcttgt | 540 |
| tgctgcgggt ggtgttgctg gtcacacgaa taaactgact aaaatgtccc cggacgtgac | 600 |
| caaaagcaac atgaccgatg acaaggcatt aaattatgcg gcacaacagg cggcgagtct | 660 |
| cggtagccag cttcagtcgc gatctctgaa cggcgattac gcgaaagata ccgctcttgg | 720 |
| tatcgctggt aaccaggctt cgtcacagtt gcaggcctgg ttacaacatt atggaacggc | 780 |
| agaggttaat ctgcaaagtg gtaataactt tgacggtagt tcactggact tcttattacc | 840 |
| gttctatgat tccgaaaaaa tgctggcatt tggtcaggtc ggagcgcgtt acattgactc | 900 |
| ccgctttacg gcaaatttag gtgcgggtca gcgttttttc cttcctgcaa acatgttggg | 960 |
| ctataacgtc ttcattgatc aggatttttc tggtgataat acccgtttag gtattggtgg | 1020 |
| cgaatactgg cgagactatt tcaaaagtag cgttaacggc tatttccgca tgagcggctg | 1080 |
| gcatgagtca tacaataaga aagactatga tgagcgccca gcaaatggct tcgatatccg | 1140 |
| ttttaatggc tatctaccgt catatccggc attaggcgcc aagctgatat atgagcagta | 1200 |
| ttatggtgat aatgttgctt tgtttaattc tgataagctg caatcgaatc ctggtgcggc | 1260 |
| gaccgttggt gtaaactata ctccgattcc tctggtgacg atggggatcg attaccgtca | 1320 |
| tggtacgggt aatgaaaatg atctccttta ctcaatgcag ttccgttatc agtttgataa | 1380 |
| atcgtggtct cagcaaattg aaccacagta tgttaacgag ttaagaacat tatcaggcag | 1440 |
| ccgttacgat ctggttcagc gtaataacaa tattattctg gagtacaaga agcaggatat | 1500 |
| tctttctctg aatattccgc atgatattaa tggtactgaa cacagtacgc agaagattca | 1560 |
| gttgatcgtt aagagcaaat acggtctgga tcgtatcgtc tgggatgata gtgcattacg | 1620 |
| cagtcagggc ggtcagattc agcatagcgg aagccaaagc gcacaagact accaggctat | 1680 |
| tttgcctgct tatgtgcaag gtggcagcaa tatttataaa gtgacggctc gcgcctatga | 1740 |
| ccgtaatggc aatagctcta acaatgtaca gcttactatt accgttctgt cgaatggtca | 1800 |
| agttgtcgac caggttgggg taacggactt tacggcggat aagacttcgg ctaaagcgga | 1860 |
| taacgccgat accattactt ataccgcgac ggtgaaaaag aatggggtag ctcaggctaa | 1920 |
| tgtccctgtt tcatttaata ttgtttcagg aactgcaact cttggggcaa atagtgccaa | 1980 |
| aacggatgct aacggtaagg caaccgtaac gttgaagtcg agtacgccag gacaggtcgt | 2040 |
| cgtgtctgct aaaaccgcgg agatgacttc agcacttaat gccagtgcgg ttatattttt | 2100 |
| tgatggtgcg cccgggaagc ttgaaaacct gtacttccaa ggtacgcgtc aggtgcagct | 2160 |
| ggtggaaacc ggcggcggcc tggtgcagcc gggcggcagc ctgcgcctga gctgcgcggc | 2220 |
| gagcggcttt acctttagca gcgtgtatat gaactgggtg cgccaggcgc cgggcaaagg | 2280 |
| cccggaatgg gtgagccgca ttagcccgaa cagcggcaac attggctata ccgatagcgt | 2340 |
| gaaaggccgc tttaccatta gccgcgataa cgcgaaaaac accctgtatc tgcagatgaa | 2400 |
| caacctgaaa ccggaagata ccgcgctgta ttattgcgcg attggcctga acctgagcag | 2460 |
| cagcagcgtg cgcggccagg gcacccaggt gaccgtgagc agcggatccg aaaacctgta | 2520 |
| cttccaaggt gactacaagg acgatgacga taagtggagc catccgcagt ttgagaaatc | 2580 |
| tagataaggt accactcgag taaggatctc caggcatcaa ataaaacgaa aggctcagtc | 2640 |
| gaaagactgg gcctttcgtt ttatctgttg tttgtcggtg aacgctctct actagagtca | 2700 |
| cactggctca ccttcgggtg ggcctttctg cgtttatacc tagggtacgg gttttgctgc | 2760 |
| ccgcaaacgg gctgttctgg tgttgctagt ttgttatcag aatcgcagat ccggcttcag | 2820 |
| ccggtttgcc ggctgaaagc gctatttctt ccagaattgc catgattttt tccccacggg | 2880 |
| aggcgtcact ggctcccgtg ttgtcggcag ctttgattcg ataagcagca tcgcctgttt | 2940 |
| caggctgtct atgtgtgact gttgagctgt aacaagttgt ctcaggtgtt caatttcatg | 3000 |
| ttctagttgc tttgttttac tggtttcacc tgttctatta ggtgttacat gctgttcatc | 3060 |
| tgttacattg tcgatctgtt catggtgaac agctttgaat gcaccaaaaa ctcgtaaaag | 3120 |
| ctctgatgta tctatctttt ttacaccgtt ttcatctgtg catatggaca gttttccctt | 3180 |
| tgatatgtaa cggtgaacag ttgttctact tttgtttgtt agtcttgatg cttcactgat | 3240 |
| agatacaaga gccataagaa cctcagatcc ttccgtattt agccagtatg ttctctagtg | 3300 |
| tggttcgttg tttttgcgtg agccatgaga acgaaccatt gagatcatac ttactttgca | 3360 |
| tgtcactcaa aaattttgcc tcaaaactgg tgagctgaat ttttgcagtt aaagcatcgt | 3420 |
| gtagtgtttt tcttagtccg ttatgtaggt aggaatctga tgtaatggtt gttggtattt | 3480 |
| tgtcaccatt catttttatc tggttgttct caagttcggt tacgagatcc atttgtctat | 3540 |
| ctagttcaac ttggaaaatc aacgtatcag tcgggcggcc tcgcttatca accaccaatt | 3600 |
| tcatattgct gtaagtgttt aaatctttac ttattggttt caaaacccat tggttaagcc | 3660 |
| ttttaaactc atggtagtta ttttcaagca ttaacatgaa cttaaattca tcaaggctaa | 3720 |
| tctctatatt tgccttgtga gttttctttt gtgttagttc ttttaataac cactcataaa | 3780 |
| tcctcataga gtatttgttt tcaaaagact taacatgttc cagattatat tttatgaatt | 3840 |
| tttttaactg gaaaagataa ggcaatatct cttcactaaa aactaattct aatttttcgc | 3900 |
| ttgagaactt ggcatagttt gtccactgga aaatctcaaa gcctttaacc aaaggattcc | 3960 |
| tgatttccac agttctcgtc atcagctctc tggttgcttt agctaataca ccataagcat | 4020 |
| tttccctact gatgttcatc atctgagcgt attggttata agtgaacgat accgtccgtt | 4080 |
| ctttccttgt agggttttca atcgtggggt tgagtagtgc cacacagcat aaaattagct | 4140 |
| tggtttcatg ctccgttaag tcatagcgac taatcgctag ttcatttgct ttgaaaacaa | 4200 |
| ctaattcaga catacatctc aattggtcta ggtgatttta atcactatac caattgagat | 4260 |
| gggctagtca atgataatta ctagtccttt tcccgggtga tctgggtatc tgtaaattct | 4320 |
| gctagacctt tgctggaaaa cttgtaaatt ctgctagacc ctctgtaaat tccgctagac | 4380 |
| ctttgtgtgt tttttttgtt tatattcaag tggttataat ttatagaata aagaaagaat | 4440 |
| aaaaaaagat aaaaagaata gatcccagcc ctgtgtataa ctcactactt tagtcagttc | 4500 |
| cgcagtatta caaaaggatg tcgcaaacgc tgtttgctcc tctacaaaac agaccttaaa | 4560 |
| accctaaagg cttaagtagc accctcgcaa gctcgggcaa atcgctgaat attccttttg | 4620 |
| tctccgacca tcaggcacct gagtcgctgt ctttttcgtg acattcagtt cgctgcgctc | 4680 |
| acggctctgg cagtgaatgg gggtaaatgg cactacaggc gccttttatg gattcatgca | 4740 |
| aggaaactac ccataataca agaaaagccc gtcacgggct tctcagggcg ttttatggcg | 4800 |
| ggtctgctat gtggtgctat ctgacttttt gctgttcagc agttcctgcc ctctgatttt | 4860 |
| ccagtctgac cacttcggat tatcccgtga caggtcattc agactggcta atgcacccag | 4920 |
| taaggcagcg gtatcatcaa caggcttacc cgtcttactg tccctagtgc ttggattctc | 4980 |
| accaataaaa aacgcccggc ggcaaccgag cgttctgaac aaatccagat ggagttctga | 5040 |
| ggtcattact ggatctatca acaggagtcc aagcgagctc gatatcaaat tacgccccgc | 5100 |
| cctgccactc atcgcagtac tgttgtaatt cattaagcat tctgccgaca tggaagccat | 5160 |
| cacaaacggc atgatgaacc tgaatcgcca gcggcatcag caccttgtcg ccttgcgtat | 5220 |
| aatatttgcc catggtgaaa acgggggcga agaagttgtc catattggcc acgtttaaat | 5280 |
| caaaactggt gaaactcacc cagggattgg ctgagacgaa aaacatattc tcaataaacc | 5340 |
| ctttagggaa ataggccagg ttttcaccgt aacacgccac atcttgcgaa tatatgtgta | 5400 |
| gaaactgccg gaaatcgtcg tggtattcac tccagagcga tgaaaacgtt tcagtttgct | 5460 |
| catggaaaac ggtgtaacaa gggtgaacac tatcccatat caccagctca ccgtctttca | 5520 |
| ttgccatacg aaattccgga tgagcattca tcaggcgggc aagaatgtga ataaaggccg | 5580 |
| gataaaactt gtgcttattt ttctttacgg tctttaaaaa ggccgtaata tccagctgaa | 5640 |
| cggtctggtt ataggtacat tgagcaactg actgaaatgc ctcaaaatgt tctttacgat | 5700 |
| gccattggga tatatcaacg gtggtatatc cagtgatttt tttctccatt ttagcttcct | 5760 |
| tagctcctga aaatctcgat aactcaaaaa atacgcccgg tagtgatctt atttcattat | 5820 |
| ggtgaaagtt ggaacctctt acgtgccgat caacgtctca ttttcgccag atatc | 5875 |
| SEQ ID NO: 6 | |
| gacgtcttga cagctagctc agtcctaggg attgtgctag caggtttaat cgaattgacg | 60 |
| tctttacggc tagctcagtc ctaggtacta tgctagcagg tttaatcgaa ttcaaaagat | 120 |
| cttttaagaa ggagatatac atatgattac tcatggttgt tatacccgga cccggcacaa | 180 |
| gcataagcta aaaaaaacat tgattatgct tagtgctggt ttaggattgt ttttttatgt | 240 |
| taatcagaac tcatttgcaa atggtgaaaa ttattttaaa ttgggttcgg attcaaaact | 300 |
| gttaactcat gatagctatc agaatcgcct tttttatacg ttgaaaactg gtgaaactgt | 360 |
| tgccgatctt tctaaatcgc aagatattaa tttatcgacg atttggtcgt tgaataagca | 420 |
| tttatacagt tctgaaagcg aaatgatgaa ggccgcgcct ggtcagcaga tcattttgcc | 480 |
| actcaaaaaa cttccctttg aatacagtgc actaccactt ttaggttcgg cacctcttgt | 540 |
| tgctgcgggt ggtgttgctg gtcacacgaa taaactgact aaaatgtccc cggacgtgac | 600 |
| caaaagcaac atgaccgatg acaaggcatt aaattatgcg gcacaacagg cggcgagtct | 660 |
| cggtagccag cttcagtcgc gatctctgaa cggcgattac gcgaaagata ccgctcttgg | 720 |
| tatcgctggt aaccaggctt cgtcacagtt gcaggcctgg ttacaacatt atggaacggc | 780 |
| agaggttaat ctgcaaagtg gtaataactt tgacggtagt tcactggact tcttattacc | 840 |
| gttctatgat tccgaaaaaa tgctggcatt tggtcaggtc ggagcgcgtt acattgactc | 900 |
| ccgctttacg gcaaatttag gtgcgggtca gcgttttttc cttcctgcaa acatgttggg | 960 |
| ctataacgtc ttcattgatc aggatttttc tggtgataat acccgtttag gtattggtgg | 1020 |
| cgaatactgg cgagactatt tcaaaagtag cgttaacggc tatttccgca tgagcggctg | 1080 |
| gcatgagtca tacaataaga aagactatga tgagcgccca gcaaatggct tcgatatccg | 1140 |
| ttttaatggc tatctaccgt catatccggc attaggcgcc aagctgatat atgagcagta | 1200 |
| ttatggtgat aatgttgctt tgtttaattc tgataagctg caatcgaatc ctggtgcggc | 1260 |
| gaccgttggt gtaaactata ctccgattcc tctggtgacg atggggatcg attaccgtca | 1320 |
| tggtacgggt aatgaaaatg atctccttta ctcaatgcag ttccgttatc agtttgataa | 1380 |
| atcgtggtct cagcaaattg aaccacagta tgttaacgag ttaagaacat tatcaggcag | 1440 |
| ccgttacgat ctggttcagc gtaataacaa tattattctg gagtacaaga agcaggatat | 1500 |
| tctttctctg aatattccgc atgatattaa tggtactgaa cacagtacgc agaagattca | 1560 |
| gttgatcgtt aagagcaaat acggtctgga tcgtatcgtc tgggatgata gtgcattacg | 1620 |
| cagtcagggc ggtcagattc agcatagcgg aagccaaagc gcacaagact accaggctat | 1680 |
| tttgcctgct tatgtgcaag gtggcagcaa tatttataaa gtgacggctc gcgcctatga | 1740 |
| ccgtaatggc aatagctcta acaatgtaca gcttactatt accgttctgt cgaatggtca | 1800 |
| agttgtcgac caggttgggg taacggactt tacggcggat aagacttcgg ctaaagcgga | 1860 |
| taacgccgat accattactt ataccgcgac ggtgaaaaag aatggggtag ctcaggctaa | 1920 |
| tgtccctgtt tcatttaata ttgtttcagg aactgcaact cttggggcaa atagtgccaa | 1980 |
| aacggatgct aacggtaagg caaccgtaac gttgaagtcg agtacgccag gacaggtcgt | 2040 |
| cgtgtctgct aaaaccgcgg agatgacttc agcacttaat gccagtgcgg ttatattttt | 2100 |
| tgatggtgcg cccgggaagc ttgaaaacct gtattttcag ggcacccgtc aggttcagct | 2160 |
| ggttgaaacc ggtggtggtc tggttcagcc tggtggtagc ctgcgtctga gctgtgcagc | 2220 |
| aagcggtttt acctttagca gcgtttatat gaattgggtt cgtcaggcac ctggtaaagg | 2280 |
| tccggaatgg gttagccgta ttagcccgaa tagcggtaat attggttata ccgatagcgt | 2340 |
| gaaaggtcgc tttaccatta gccgtgataa tgcaaaaaat accctgtacc tgcagatgaa | 2400 |
| taatctgaaa ccggaagata ccgcactgta ttattgtgca attggtctga atctgagcag | 2460 |
| cagcagcgtt cgtggtcagg gtacacaggt taccgtgagc agcggtagcg agaatctgta | 2520 |
| tttccaaggt gattataaag acgacgatga caagtggtcc catccgcagt ttgaaaaaag | 2580 |
| ccgttaaggt accactcgag taaggatctc caggcatcaa ataaaacgaa aggctcagtc | 2640 |
| gaaagactgg gcctttcgtt ttatctgttg tttgtcggtg aacgctctct actagagtca | 2700 |
| cactggctca ccttcgggtg ggcctttctg cgtttatacc tagggtacgg gttttgctgc | 2760 |
| ccgcaaacgg gctgttctgg tgttgctagt ttgttatcag aatcgcagat ccggcttcag | 2820 |
| ccggtttgcc ggctgaaagc gctatttctt ccagaattgc catgattttt tccccacggg | 2880 |
| aggcgtcact ggctcccgtg ttgtcggcag ctttgattcg ataagcagca tcgcctgttt | 2940 |
| caggctgtct atgtgtgact gttgagctgt aacaagttgt ctcaggtgtt caatttcatg | 3000 |
| ttctagttgc tttgttttac tggtttcacc tgttctatta ggtgttacat gctgttcatc | 3060 |
| tgttacattg tcgatctgtt catggtgaac agctttgaat gcaccaaaaa ctcgtaaaag | 3120 |
| ctctgatgta tctatctttt ttacaccgtt ttcatctgtg catatggaca gttttccctt | 3180 |
| tgatatgtaa cggtgaacag ttgttctact tttgtttgtt agtcttgatg cttcactgat | 3240 |
| agatacaaga gccataagaa cctcagatcc ttccgtattt agccagtatg ttctctagtg | 3300 |
| tggttcgttg tttttgcgtg agccatgaga acgaaccatt gagatcatac ttactttgca | 3360 |
| tgtcactcaa aaattttgcc tcaaaactgg tgagctgaat ttttgcagtt aaagcatcgt | 3420 |
| gtagtgtttt tcttagtccg ttatgtaggt aggaatctga tgtaatggtt gttggtattt | 3480 |
| tgtcaccatt catttttatc tggttgttct caagttcggt tacgagatcc atttgtctat | 3540 |
| ctagttcaac ttggaaaatc aacgtatcag tcgggcggcc tcgcttatca accaccaatt | 3600 |
| tcatattgct gtaagtgttt aaatctttac ttattggttt caaaacccat tggttaagcc | 3660 |
| ttttaaactc atggtagtta ttttcaagca ttaacatgaa cttaaattca tcaaggctaa | 3720 |
| tctctatatt tgccttgtga gttttctttt gtgttagttc ttttaataac cactcataaa | 3780 |
| tcctcataga gtatttgttt tcaaaagact taacatgttc cagattatat tttatgaatt | 3840 |
| tttttaactg gaaaagataa ggcaatatct cttcactaaa aactaattct aatttttcgc | 3900 |
| ttgagaactt ggcatagttt gtccactgga aaatctcaaa gcctttaacc aaaggattcc | 3960 |
| tgatttccac agttctcgtc atcagctctc tggttgcttt agctaataca ccataagcat | 4020 |
| tttccctact gatgttcatc atctgagcgt attggttata agtgaacgat accgtccgtt | 4080 |
| ctttccttgt agggttttca atcgtggggt tgagtagtgc cacacagcat aaaattagct | 4140 |
| tggtttcatg ctccgttaag tcatagcgac taatcgctag ttcatttgct ttgaaaacaa | 4200 |
| ctaattcaga catacatctc aattggtcta ggtgatttta atcactatac caattgagat | 4260 |
| gggctagtca atgataatta ctagtccttt tcccgggtga tctgggtatc tgtaaattct | 4320 |
| gctagacctt tgctggaaaa cttgtaaatt ctgctagacc ctctgtaaat tccgctagac | 4380 |
| ctttgtgtgt tttttttgtt tatattcaag tggttataat ttatagaata aagaaagaat | 4440 |
| aaaaaaagat aaaaagaata gatcccagcc ctgtgtataa ctcactactt tagtcagttc | 4500 |
| cgcagtatta caaaaggatg tcgcaaacgc tgtttgctcc tctacaaaac agaccttaaa | 4560 |
| accctaaagg cttaagtagc accctcgcaa gctcgggcaa atcgctgaat attccttttg | 4620 |
| tctccgacca tcaggcacct gagtcgctgt ctttttcgtg acattcagtt cgctgcgctc | 4680 |
| acggctctgg cagtgaatgg gggtaaatgg cactacaggc gccttttatg gattcatgca | 4740 |
| aggaaactac ccataataca agaaaagccc gtcacgggct tctcagggcg ttttatggcg | 4800 |
| ggtctgctat gtggtgctat ctgacttttt gctgttcagc agttcctgcc ctctgatttt | 4860 |
| ccagtctgac cacttcggat tatcccgtga caggtcattc agactggcta atgcacccag | 4920 |
| taaggcagcg gtatcatcaa caggcttacc cgtcttactg tccctagtgc ttggattctc | 4980 |
| accaataaaa aacgcccggc ggcaaccgag cgttctgaac aaatccagat ggagttctga | 5040 |
| ggtcattact ggatctatca acaggagtcc aagcgagctc gatatcaaat tacgccccgc | 5100 |
| cctgccactc atcgcagtac tgttgtaatt cattaagcat tctgccgaca tggaagccat | 5160 |
| cacaaacggc atgatgaacc tgaatcgcca gcggcatcag caccttgtcg ccttgcgtat | 5220 |
| aatatttgcc catggtgaaa acgggggcga agaagttgtc catattggcc acgtttaaat | 5280 |
| caaaactggt gaaactcacc cagggattgg ctgagacgaa aaacatattc tcaataaacc | 5340 |
| ctttagggaa ataggccagg ttttcaccgt aacacgccac atcttgcgaa tatatgtgta | 5400 |
| gaaactgccg gaaatcgtcg tggtattcac tccagagcga tgaaaacgtt tcagtttgct | 5460 |
| catggaaaac ggtgtaacaa gggtgaacac tatcccatat caccagctca ccgtctttca | 5520 |
| ttgccatacg aaattccgga tgagcattca tcaggcgggc aagaatgtga ataaaggccg | 5580 |
| gataaaactt gtgcttattt ttctttacgg tctttaaaaa ggccgtaata tccagctgaa | 5640 |
| cggtctggtt ataggtacat tgagcaactg actgaaatgc ctcaaaatgt tctttacgat | 5700 |
| gccattggga tatatcaacg gtggtatatc cagtgatttt tttctccatt ttagcttcct | 5760 |
| tagctcctga aaatctcgat aactcaaaaa atacgcccgg tagtgatctt atttcattat | 5820 |
| ggtgaaagtt ggaacctctt acgtgccgat caacgtctca ttttcgccag atatc | 5875 |
| SEQ ID NO: 7 | |
| gacgtcttga cagctagctc agtcctaggg attgtgctag caggtttaat cgaattgacg | 60 |
| tctttacggc tagctcagtc ctaggtacta tgctagcagg tttaatcgaa ttcaaaagat | 120 |
| cttttaagaa ggagatatac atatgattac tcatggttgt tatacccgga cccggcacaa | 180 |
| gcataagcta aaaaaaacat tgattatgct tagtgctggt ttaggattgt ttttttatgt | 240 |
| taatcagaac tcatttgcaa atggtgaaaa ttattttaaa ttgggttcgg attcaaaact | 300 |
| gttaactcat gatagctatc agaatcgcct tttttatacg ttgaaaactg gtgaaactgt | 360 |
| tgccgatctt tctaaatcgc aagatattaa tttatcgacg atttggtcgt tgaataagca | 420 |
| tttatacagt tctgaaagcg aaatgatgaa ggccgcgcct ggtcagcaga tcattttgcc | 480 |
| actcaaaaaa cttccctttg aatacagtgc actaccactt ttaggttcgg cacctcttgt | 540 |
| tgctgcgggt ggtgttgctg gtcacacgaa taaactgact aaaatgtccc cggacgtgac | 600 |
| caaaagcaac atgaccgatg acaaggcatt aaattatgcg gcacaacagg cggcgagtct | 660 |
| cggtagccag cttcagtcgc gatctctgaa cggcgattac gcgaaagata ccgctcttgg | 720 |
| tatcgctggt aaccaggctt cgtcacagtt gcaggcctgg ttacaacatt atggaacggc | 780 |
| agaggttaat ctgcaaagtg gtaataactt tgacggtagt tcactggact tcttattacc | 840 |
| gttctatgat tccgaaaaaa tgctggcatt tggtcaggtc ggagcgcgtt acattgactc | 900 |
| ccgctttacg gcaaatttag gtgcgggtca gcgttttttc cttcctgcaa acatgttggg | 960 |
| ctataacgtc ttcattgatc aggatttttc tggtgataat acccgtttag gtattggtgg | 1020 |
| cgaatactgg cgagactatt tcaaaagtag cgttaacggc tatttccgca tgagcggctg | 1080 |
| gcatgagtca tacaataaga aagactatga tgagcgccca gcaaatggct tcgatatccg | 1140 |
| ttttaatggc tatctaccgt catatccggc attaggcgcc aagctgatat atgagcagta | 1200 |
| ttatggtgat aatgttgctt tgtttaattc tgataagctg caatcgaatc ctggtgcggc | 1260 |
| gaccgttggt gtaaactata ctccgattcc tctggtgacg atggggatcg attaccgtca | 1320 |
| tggtacgggt aatgaaaatg atctccttta ctcaatgcag ttccgttatc agtttgataa | 1380 |
| atcgtggtct cagcaaattg aaccacagta tgttaacgag ttaagaacat tatcaggcag | 1440 |
| ccgttacgat ctggttcagc gtaataacaa tattattctg gagtacaaga agcaggatat | 1500 |
| tctttctctg aatattccgc atgatattaa tggtactgaa cacagtacgc agaagattca | 1560 |
| gttgatcgtt aagagcaaat acggtctgga tcgtatcgtc tgggatgata gtgcattacg | 1620 |
| cagtcagggc ggtcagattc agcatagcgg aagccaaagc gcacaagact accaggctat | 1680 |
| tttgcctgct tatgtgcaag gtggcagcaa tatttataaa gtgacggctc gcgcctatga | 1740 |
| ccgtaatggc aatagctcta acaatgtaca gcttactatt accgttctgt cgaatggtca | 1800 |
| agttgtcgac caggttgggg taacggactt tacggcggat aagacttcgg ctaaagcgga | 1860 |
| taacgccgat accattactt ataccgcgac ggtgaaaaag aatggggtag ctcaggctaa | 1920 |
| tgtccctgtt tcatttaata ttgtttcagg aactgcaact cttggggcaa atagtgccaa | 1980 |
| aacggatgct aacggtaagg caaccgtaac gttgaagtcg agtacgccag gacaggtcgt | 2040 |
| cgtgtctgct aaaaccgcgg agatgacttc agcacttaat gccagtgcgg ttatattttt | 2100 |
| tgatggtgcg cccgggaagc ttgaaaacct gtacttccaa ggtacgcgtc aggtgcagct | 2160 |
| gcaggaaagc ggtggtggtc tggtgcaggc cggtggtagc ctgcgtctga gctgtgccgc | 2220 |
| cagcggtcgt acctttagcg aatatgccat gggttggttt cgtcaggccc cgggtaaaga | 2280 |
| acgtgaattt gtggccacca ttagctggag cggtggtagc acctattata ccgatagcgt | 2340 |
| gaaaggtcgt tttaccatta gccgtgataa tgccaaaaat accgtgtatc tgcagatgaa | 2400 |
| tagcctgaaa ccggatgata ccgccgtgta ttattgtgcc gccgccggtc tgggtaccgt | 2460 |
| ggtgagcgaa tgggattatg attatgatta ttggggtcag ggtacccagg tgaccgtgag | 2520 |
| cagcggtagc ggatccgaaa acctgtactt ccaaggtgac tacaaggacg atgacgataa | 2580 |
| gtggagccat ccgcagtttg agaaatctag ataaggtacc actcgagtaa ggatctccag | 2640 |
| gcatcaaata aaacgaaagg ctcagtcgaa agactgggcc tttcgtttta tctgttgttt | 2700 |
| gtcggtgaac gctctctact agagtcacac tggctcacct tcgggtgggc ctttctgcgt | 2760 |
| ttatacctag ggtacgggtt ttgctgcccg caaacgggct gttctggtgt tgctagtttg | 2820 |
| ttatcagaat cgcagatccg gcttcagccg gtttgccggc tgaaagcgct atttcttcca | 2880 |
| gaattgccat gattttttcc ccacgggagg cgtcactggc tcccgtgttg tcggcagctt | 2940 |
| tgattcgata agcagcatcg cctgtttcag gctgtctatg tgtgactgtt gagctgtaac | 3000 |
| aagttgtctc aggtgttcaa tttcatgttc tagttgcttt gttttactgg tttcacctgt | 3060 |
| tctattaggt gttacatgct gttcatctgt tacattgtcg atctgttcat ggtgaacagc | 3120 |
| tttgaatgca ccaaaaactc gtaaaagctc tgatgtatct atctttttta caccgttttc | 3180 |
| atctgtgcat atggacagtt ttccctttga tatgtaacgg tgaacagttg ttctactttt | 3240 |
| gtttgttagt cttgatgctt cactgataga tacaagagcc ataagaacct cagatccttc | 3300 |
| cgtatttagc cagtatgttc tctagtgtgg ttcgttgttt ttgcgtgagc catgagaacg | 3360 |
| aaccattgag atcatactta ctttgcatgt cactcaaaaa ttttgcctca aaactggtga | 3420 |
| gctgaatttt tgcagttaaa gcatcgtgta gtgtttttct tagtccgtta tgtaggtagg | 3480 |
| aatctgatgt aatggttgtt ggtattttgt caccattcat ttttatctgg ttgttctcaa | 3540 |
| gttcggttac gagatccatt tgtctatcta gttcaacttg gaaaatcaac gtatcagtcg | 3600 |
| ggcggcctcg cttatcaacc accaatttca tattgctgta agtgtttaaa tctttactta | 3660 |
| ttggtttcaa aacccattgg ttaagccttt taaactcatg gtagttattt tcaagcatta | 3720 |
| acatgaactt aaattcatca aggctaatct ctatatttgc cttgtgagtt ttcttttgtg | 3780 |
| ttagttcttt taataaccac tcataaatcc tcatagagta tttgttttca aaagacttaa | 3840 |
| catgttccag attatatttt atgaattttt ttaactggaa aagataaggc aatatctctt | 3900 |
| cactaaaaac taattctaat ttttcgcttg agaacttggc atagtttgtc cactggaaaa | 3960 |
| tctcaaagcc tttaaccaaa ggattcctga tttccacagt tctcgtcatc agctctctgg | 4020 |
| ttgctttagc taatacacca taagcatttt ccctactgat gttcatcatc tgagcgtatt | 4080 |
| ggttataagt gaacgatacc gtccgttctt tccttgtagg gttttcaatc gtggggttga | 4140 |
| gtagtgccac acagcataaa attagcttgg tttcatgctc cgttaagtca tagcgactaa | 4200 |
| tcgctagttc atttgctttg aaaacaacta attcagacat acatctcaat tggtctaggt | 4260 |
| gattttaatc actataccaa ttgagatggg ctagtcaatg ataattacta gtccttttcc | 4320 |
| cgggtgatct gggtatctgt aaattctgct agacctttgc tggaaaactt gtaaattctg | 4380 |
| ctagaccctc tgtaaattcc gctagacctt tgtgtgtttt ttttgtttat attcaagtgg | 4440 |
| ttataattta tagaataaag aaagaataaa aaaagataaa aagaatagat cccagccctg | 4500 |
| tgtataactc actactttag tcagttccgc agtattacaa aaggatgtcg caaacgctgt | 4560 |
| ttgctcctct acaaaacaga ccttaaaacc ctaaaggctt aagtagcacc ctcgcaagct | 4620 |
| cgggcaaatc gctgaatatt ccttttgtct ccgaccatca ggcacctgag tcgctgtctt | 4680 |
| tttcgtgaca ttcagttcgc tgcgctcacg gctctggcag tgaatggggg taaatggcac | 4740 |
| tacaggcgcc ttttatggat tcatgcaagg aaactaccca taatacaaga aaagcccgtc | 4800 |
| acgggcttct cagggcgttt tatggcgggt ctgctatgtg gtgctatctg actttttgct | 4860 |
| gttcagcagt tcctgccctc tgattttcca gtctgaccac ttcggattat cccgtgacag | 4920 |
| gtcattcaga ctggctaatg cacccagtaa ggcagcggta tcatcaacag gcttacccgt | 4980 |
| cttactgtcc ctagtgcttg gattctcacc aataaaaaac gcccggcggc aaccgagcgt | 5040 |
| tctgaacaaa tccagatgga gttctgaggt cattactgga tctatcaaca ggagtccaag | 5100 |
| cgagctcgat atcaaattac gccccgccct gccactcatc gcagtactgt tgtaattcat | 5160 |
| taagcattct gccgacatgg aagccatcac aaacggcatg atgaacctga atcgccagcg | 5220 |
| gcatcagcac cttgtcgcct tgcgtataat atttgcccat ggtgaaaacg ggggcgaaga | 5280 |
| agttgtccat attggccacg tttaaatcaa aactggtgaa actcacccag ggattggctg | 5340 |
| agacgaaaaa catattctca ataaaccctt tagggaaata ggccaggttt tcaccgtaac | 5400 |
| acgccacatc ttgcgaatat atgtgtagaa actgccggaa atcgtcgtgg tattcactcc | 5460 |
| agagcgatga aaacgtttca gtttgctcat ggaaaacggt gtaacaaggg tgaacactat | 5520 |
| cccatatcac cagctcaccg tctttcattg ccatacgaaa ttccggatga gcattcatca | 5580 |
| ggcgggcaag aatgtgaata aaggccggat aaaacttgtg cttatttttc tttacggtct | 5640 |
| ttaaaaaggc cgtaatatcc agctgaacgg tctggttata ggtacattga gcaactgact | 5700 |
| gaaatgcctc aaaatgttct ttacgatgcc attgggatat atcaacggtg gtatatccag | 5760 |
| tgattttttt ctccatttta gcttccttag ctcctgaaaa tctcgataac tcaaaaaata | 5820 |
| cgcccggtag tgatcttatt tcattatggt gaaagttgga acctcttacg tgccgatcaa | 5880 |
| cgtctcattt tcgccagata tc | 5902 |
| SEQ ID NO: 8 | |
| gacgtcttga cagctagctc agtcctaggg attgtgctag caggtttaat cgaattgacg | 60 |
| tctttacggc tagctcagtc ctaggtacta tgctagcagg tttaatcgaa ttcaaaagat | 120 |
| cttttaagaa ggagatatac atatgaaagc aaccaagctg gttctgggtg ccgtgattct | 180 |
| gggcagtacc ctgttagcag gttgttctag caatgccaaa atcgaccaag gcatcaacaa | 240 |
| caatggcccg acccacgaaa accagctggg tgccggtgcc tttggtggtt atcaggtgaa | 300 |
| cccgtacgtg ggctttgaaa tgggctatga ttggctgggc cgcatgccgt acaaaggcag | 360 |
| tgtggagaac ggcgcctata aagcacaggg cgtgcagctg acagcaaaac tgggctaccc | 420 |
| tattaccgac gacctggaca tctacacacg cttaggcggc atggtgtggc gcgccgatac | 480 |
| caagagcaac gtgtacggca agaaccacga taccggcgtg agtccggtgt ttgccggcgg | 540 |
| tgtggagtat gcaatcaccc cggaaattgc cacacgtaag cttgaaaacc tgtacttcca | 600 |
| aggtacgcgt caggtgcagc tggtggaaac cggcggcggc ctggtgcagc cgggcggcag | 660 |
| cctgcgcctg agctgcgcgg cgagcggctt tacctttagc agcgtgtata tgaactgggt | 720 |
| gcgccaggcg ccgggcaaag gcccggaatg ggtgagccgc attagcccga acagcggcaa | 780 |
| cattggctat accgatagcg tgaaaggccg ctttaccatt agccgcgata acgcgaaaaa | 840 |
| caccctgtat ctgcagatga acaacctgaa accggaagat accgcgctgt attattgcgc | 900 |
| gattggcctg aacctgagca gcagcagcgt gcgcggccag ggcacccagg tgaccgtgag | 960 |
| cagcggatcc gaaaacctgt acttccaagg tgactacaag gacgatgacg ataagtggag | 1020 |
| ccatccgcag tttgagaaat ctagataagg taccactcga gtaaggatct ccaggcatca | 1080 |
| aataaaacga aaggctcagt cgaaagactg ggcctttcgt tttatctgtt gtttgtcggt | 1140 |
| gaacgctctc tactagagtc acactggctc accttcgggt gggcctttct gcgtttatac | 1200 |
| ctagggtacg ggttttgctg cccgcaaacg ggctgttctg gtgttgctag tttgttatca | 1260 |
| gaatcgcaga tccggcttca gccggtttgc cggctgaaag cgctatttct tccagaattg | 1320 |
| ccatgatttt ttccccacgg gaggcgtcac tggctcccgt gttgtcggca gctttgattc | 1380 |
| gataagcagc atcgcctgtt tcaggctgtc tatgtgtgac tgttgagctg taacaagttg | 1440 |
| tctcaggtgt tcaatttcat gttctagttg ctttgtttta ctggtttcac ctgttctatt | 1500 |
| aggtgttaca tgctgttcat ctgttacatt gtcgatctgt tcatggtgaa cagctttgaa | 1560 |
| tgcaccaaaa actcgtaaaa gctctgatgt atctatcttt tttacaccgt tttcatctgt | 1620 |
| gcatatggac agttttccct ttgatatgta acggtgaaca gttgttctac ttttgtttgt | 1680 |
| tagtcttgat gcttcactga tagatacaag agccataaga acctcagatc cttccgtatt | 1740 |
| tagccagtat gttctctagt gtggttcgtt gtttttgcgt gagccatgag aacgaaccat | 1800 |
| tgagatcata cttactttgc atgtcactca aaaattttgc ctcaaaactg gtgagctgaa | 1860 |
| tttttgcagt taaagcatcg tgtagtgttt ttcttagtcc gttatgtagg taggaatctg | 1920 |
| atgtaatggt tgttggtatt ttgtcaccat tcatttttat ctggttgttc tcaagttcgg | 1980 |
| ttacgagatc catttgtcta tctagttcaa cttggaaaat caacgtatca gtcgggcggc | 2040 |
| ctcgcttatc aaccaccaat ttcatattgc tgtaagtgtt taaatcttta cttattggtt | 2100 |
| tcaaaaccca ttggttaagc cttttaaact catggtagtt attttcaagc attaacatga | 2160 |
| acttaaattc atcaaggcta atctctatat ttgccttgtg agttttcttt tgtgttagtt | 2220 |
| cttttaataa ccactcataa atcctcatag agtatttgtt ttcaaaagac ttaacatgtt | 2280 |
| ccagattata ttttatgaat ttttttaact ggaaaagata aggcaatatc tcttcactaa | 2340 |
| aaactaattc taatttttcg cttgagaact tggcatagtt tgtccactgg aaaatctcaa | 2400 |
| agcctttaac caaaggattc ctgatttcca cagttctcgt catcagctct ctggttgctt | 2460 |
| tagctaatac accataagca ttttccctac tgatgttcat catctgagcg tattggttat | 2520 |
| aagtgaacga taccgtccgt tctttccttg tagggttttc aatcgtgggg ttgagtagtg | 2580 |
| ccacacagca taaaattagc ttggtttcat gctccgttaa gtcatagcga ctaatcgcta | 2640 |
| gttcatttgc tttgaaaaca actaattcag acatacatct caattggtct aggtgatttt | 2700 |
| aatcactata ccaattgaga tgggctagtc aatgataatt actagtcctt ttcccgggtg | 2760 |
| atctgggtat ctgtaaattc tgctagacct ttgctggaaa acttgtaaat tctgctagac | 2820 |
| cctctgtaaa ttccgctaga cctttgtgtg ttttttttgt ttatattcaa gtggttataa | 2880 |
| tttatagaat aaagaaagaa taaaaaaaga taaaaagaat agatcccagc cctgtgtata | 2940 |
| actcactact ttagtcagtt ccgcagtatt acaaaaggat gtcgcaaacg ctgtttgctc | 3000 |
| ctctacaaaa cagaccttaa aaccctaaag gcttaagtag caccctcgca agctcgggca | 3060 |
| aatcgctgaa tattcctttt gtctccgacc atcaggcacc tgagtcgctg tctttttcgt | 3120 |
| gacattcagt tcgctgcgct cacggctctg gcagtgaatg ggggtaaatg gcactacagg | 3180 |
| cgccttttat ggattcatgc aaggaaacta cccataatac aagaaaagcc cgtcacgggc | 3240 |
| ttctcagggc gttttatggc gggtctgcta tgtggtgcta tctgactttt tgctgttcag | 3300 |
| cagttcctgc cctctgattt tccagtctga ccacttcgga ttatcccgtg acaggtcatt | 3360 |
| cagactggct aatgcaccca gtaaggcagc ggtatcatca acaggcttac ccgtcttact | 3420 |
| gtccctagtg cttggattct caccaataaa aaacgcccgg cggcaaccga gcgttctgaa | 3480 |
| caaatccaga tggagttctg aggtcattac tggatctatc aacaggagtc caagcgagct | 3540 |
| cgatatcaaa ttacgccccg ccctgccact catcgcagta ctgttgtaat tcattaagca | 3600 |
| ttctgccgac atggaagcca tcacaaacgg catgatgaac ctgaatcgcc agcggcatca | 3660 |
| gcaccttgtc gccttgcgta taatatttgc ccatggtgaa aacgggggcg aagaagttgt | 3720 |
| ccatattggc cacgtttaaa tcaaaactgg tgaaactcac ccagggattg gctgagacga | 3780 |
| aaaacatatt ctcaataaac cctttaggga aataggccag gttttcaccg taacacgcca | 3840 |
| catcttgcga atatatgtgt agaaactgcc ggaaatcgtc gtggtattca ctccagagcg | 3900 |
| atgaaaacgt ttcagtttgc tcatggaaaa cggtgtaaca agggtgaaca ctatcccata | 3960 |
| tcaccagctc accgtctttc attgccatac gaaattccgg atgagcattc atcaggcggg | 4020 |
| caagaatgtg aataaaggcc ggataaaact tgtgcttatt tttctttacg gtctttaaaa | 4080 |
| aggccgtaat atccagctga acggtctggt tataggtaca ttgagcaact gactgaaatg | 4140 |
| cctcaaaatg ttctttacga tgccattggg atatatcaac ggtggtatat ccagtgattt | 4200 |
| ttttctccat tttagcttcc ttagctcctg aaaatctcga taactcaaaa aatacgcccg | 4260 |
| gtagtgatct tatttcatta tggtgaaagt tggaacctct tacgtgccga tcaacgtctc | 4320 |
| attttcgcca gatatc | 4336 |
| SEQ ID NO: 9 | |
| gacgtcttga cagctagctc agtcctaggg attgtgctag caggtttaat cgaattgacg | 60 |
| tctttacggc tagctcagtc ctaggtacta tgctagcagg tttaatcgaa ttcaaaagat | 120 |
| cttttaagaa ggagatatac atatgaaagc aaccaagctg gttctgggtg ccgtgattct | 180 |
| gggcagtacc ctgttagcag gttgttctag caatgccaaa atcgaccaag gcatcaacaa | 240 |
| caatggcccg acccacgaaa accagctggg tgccggtgcc tttggtggtt atcaggtgaa | 300 |
| cccgtacgtg ggctttgaaa tgggctatga ttggctgggc cgcatgccgt acaaaggcag | 360 |
| tgtggagaac ggcgcctata aagcacaggg cgtgcagctg acagcaaaac tgggctaccc | 420 |
| tattaccgac gacctggaca tctacacacg cttaggcggc atggtgtggc gcgccgatac | 480 |
| caagagcaac gtgtacggca agaaccacga taccggcgtg agtccggtgt ttgccggcgg | 540 |
| tgtggagtat gcaatcaccc cggaaattgc cacacgtaag cttgaaaacc tgtacttcca | 600 |
| aggtacgcgt caggtgcagc tgcaggaaag cggtggtggt ctggtgcagg ccggtggtag | 660 |
| cctgcgtctg agctgtgccg ccagcggtcg tacctttagc gaatatgcca tgggttggtt | 720 |
| tcgtcaggcc ccgggtaaag aacgtgaatt tgtggccacc attagctgga gcggtggtag | 780 |
| cacctattat accgatagcg tgaaaggtcg ttttaccatt agccgtgata atgccaaaaa | 840 |
| taccgtgtat ctgcagatga atagcctgaa accggatgat accgccgtgt attattgtgc | 900 |
| cgccgccggt ctgggtaccg tggtgagcga atgggattat gattatgatt attggggtca | 960 |
| gggtacccag gtgaccgtga gcagcggtag cggatccgaa aacctgtact tccaaggtga | 1020 |
| ctacaaggac gatgacgata agtggagcca tccgcagttt gagaaatcta gataaggtac | 1080 |
| cactcgagta aggatctcca ggcatcaaat aaaacgaaag gctcagtcga aagactgggc | 1140 |
| ctttcgtttt atctgttgtt tgtcggtgaa cgctctctac tagagtcaca ctggctcacc | 1200 |
| ttcgggtggg cctttctgcg tttataccta gggtacgggt tttgctgccc gcaaacgggc | 1260 |
| tgttctggtg ttgctagttt gttatcagaa tcgcagatcc ggcttcagcc ggtttgccgg | 1320 |
| ctgaaagcgc tatttcttcc agaattgcca tgattttttc cccacgggag gcgtcactgg | 1380 |
| ctcccgtgtt gtcggcagct ttgattcgat aagcagcatc gcctgtttca ggctgtctat | 1440 |
| gtgtgactgt tgagctgtaa caagttgtct caggtgttca atttcatgtt ctagttgctt | 1500 |
| tgttttactg gtttcacctg ttctattagg tgttacatgc tgttcatctg ttacattgtc | 1560 |
| gatctgttca tggtgaacag ctttgaatgc accaaaaact cgtaaaagct ctgatgtatc | 1620 |
| tatctttttt acaccgtttt catctgtgca tatggacagt tttccctttg atatgtaacg | 1680 |
| gtgaacagtt gttctacttt tgtttgttag tcttgatgct tcactgatag atacaagagc | 1740 |
| cataagaacc tcagatcctt ccgtatttag ccagtatgtt ctctagtgtg gttcgttgtt | 1800 |
| tttgcgtgag ccatgagaac gaaccattga gatcatactt actttgcatg tcactcaaaa | 1860 |
| attttgcctc aaaactggtg agctgaattt ttgcagttaa agcatcgtgt agtgtttttc | 1920 |
| ttagtccgtt atgtaggtag gaatctgatg taatggttgt tggtattttg tcaccattca | 1980 |
| tttttatctg gttgttctca agttcggtta cgagatccat ttgtctatct agttcaactt | 2040 |
| ggaaaatcaa cgtatcagtc gggcggcctc gcttatcaac caccaatttc atattgctgt | 2100 |
| aagtgtttaa atctttactt attggtttca aaacccattg gttaagcctt ttaaactcat | 2160 |
| ggtagttatt ttcaagcatt aacatgaact taaattcatc aaggctaatc tctatatttg | 2220 |
| ccttgtgagt tttcttttgt gttagttctt ttaataacca ctcataaatc ctcatagagt | 2280 |
| atttgttttc aaaagactta acatgttcca gattatattt tatgaatttt tttaactgga | 2340 |
| aaagataagg caatatctct tcactaaaaa ctaattctaa tttttcgctt gagaacttgg | 2400 |
| catagtttgt ccactggaaa atctcaaagc ctttaaccaa aggattcctg atttccacag | 2460 |
| ttctcgtcat cagctctctg gttgctttag ctaatacacc ataagcattt tccctactga | 2520 |
| tgttcatcat ctgagcgtat tggttataag tgaacgatac cgtccgttct ttccttgtag | 2580 |
| ggttttcaat cgtggggttg agtagtgcca cacagcataa aattagcttg gtttcatgct | 2640 |
| ccgttaagtc atagcgacta atcgctagtt catttgcttt gaaaacaact aattcagaca | 2700 |
| tacatctcaa ttggtctagg tgattttaat cactatacca attgagatgg gctagtcaat | 2760 |
| gataattact agtccttttc ccgggtgatc tgggtatctg taaattctgc tagacctttg | 2820 |
| ctggaaaact tgtaaattct gctagaccct ctgtaaattc cgctagacct ttgtgtgttt | 2880 |
| tttttgttta tattcaagtg gttataattt atagaataaa gaaagaataa aaaaagataa | 2940 |
| aaagaataga tcccagccct gtgtataact cactacttta gtcagttccg cagtattaca | 3000 |
| aaaggatgtc gcaaacgctg tttgctcctc tacaaaacag accttaaaac cctaaaggct | 3060 |
| taagtagcac cctcgcaagc tcgggcaaat cgctgaatat tccttttgtc tccgaccatc | 3120 |
| aggcacctga gtcgctgtct ttttcgtgac attcagttcg ctgcgctcac ggctctggca | 3180 |
| gtgaatgggg gtaaatggca ctacaggcgc cttttatgga ttcatgcaag gaaactaccc | 3240 |
| ataatacaag aaaagcccgt cacgggcttc tcagggcgtt ttatggcggg tctgctatgt | 3300 |
| ggtgctatct gactttttgc tgttcagcag ttcctgccct ctgattttcc agtctgacca | 3360 |
| cttcggatta tcccgtgaca ggtcattcag actggctaat gcacccagta aggcagcggt | 3420 |
| atcatcaaca ggcttacccg tcttactgtc cctagtgctt ggattctcac caataaaaaa | 3480 |
| cgcccggcgg caaccgagcg ttctgaacaa atccagatgg agttctgagg tcattactgg | 3540 |
| atctatcaac aggagtccaa gcgagctcga tatcaaatta cgccccgccc tgccactcat | 3600 |
| cgcagtactg ttgtaattca ttaagcattc tgccgacatg gaagccatca caaacggcat | 3660 |
| gatgaacctg aatcgccagc ggcatcagca ccttgtcgcc ttgcgtataa tatttgccca | 3720 |
| tggtgaaaac gggggcgaag aagttgtcca tattggccac gtttaaatca aaactggtga | 3780 |
| aactcaccca gggattggct gagacgaaaa acatattctc aataaaccct ttagggaaat | 3840 |
| aggccaggtt ttcaccgtaa cacgccacat cttgcgaata tatgtgtaga aactgccgga | 3900 |
| aatcgtcgtg gtattcactc cagagcgatg aaaacgtttc agtttgctca tggaaaacgg | 3960 |
| tgtaacaagg gtgaacacta tcccatatca ccagctcacc gtctttcatt gccatacgaa | 4020 |
| attccggatg agcattcatc aggcgggcaa gaatgtgaat aaaggccgga taaaacttgt | 4080 |
| gcttattttt ctttacggtc tttaaaaagg ccgtaatatc cagctgaacg gtctggttat | 4140 |
| aggtacattg agcaactgac tgaaatgcct caaaatgttc tttacgatgc cattgggata | 4200 |
| tatcaacggt ggtatatcca gtgatttttt tctccatttt agcttcctta gctcctgaaa | 4260 |
| atctcgataa ctcaaaaaat acgcccggta gtgatcttat ttcattatgg tgaaagttgg | 4320 |
| aacctcttac gtgccgatca acgtctcatt ttcgccagat atc | 4363 |
| SEQ ID NO: 10 | |
| gacgtcttga cagctagctc agtcctaggg attgtgctag caggtttaat cgaattgacg | 60 |
| tctttacggc tagctcagtc ctaggtacta tgctagcagg tttaatcgaa ttcaaaagat | 120 |
| cttttaagaa ggagatatac atatgaaagc caccaaactg gttctgggtg cagttattct | 180 |
| gggtagcacc ctgctggcag gttgtagcag caatgcaaaa attgatcagg gcattaataa | 240 |
| caacggtccg acacatgaaa atcagttagg tgccggtgca tttggtggtt atcaggttaa | 300 |
| tccgtatgtg ggttttgaaa tgggttatga ttggctgggt cgtatgccgt ataaaggtag | 360 |
| cgttgaaaat ggtgcatata aagcacaggg tgttcagctg accgcaaaac tgggttatcc | 420 |
| gattaccgat gatctggata tctatacccg tttaggtggt atggtttggc gtgcagatac | 480 |
| caaaagcaat gtgtatggca aaaatcatga taccggtgtt agtccggttt ttgccggtgg | 540 |
| tgttgaatat gcaattacac cggaaattgc aacccgcaaa ctggaaaatc tgtattttca | 600 |
| gggcacccgt caggttcagc tgcaagaaag cggtggtggt ctggttcagg caggcggtag | 660 |
| cctgcgtctg agctgtgcag caagcggtcg tacctttagc gaatatgcca tgggttggtt | 720 |
| tcgtcaggca ccgggtaaag aacgtgaatt tgttgcaacc attagctggt ctggtggtag | 780 |
| cacctattat accgatagcg ttaaaggtcg ttttaccatt agccgtgata atgccaaaaa | 840 |
| taccgtttac ctgcagatga atagcctgaa accggatgat accgcagtgt attattgtgc | 900 |
| agcagcaggt ctgggtacag ttgttagcga gtgggattat gattatgact attggggtca | 960 |
| gggtacacag gttaccgtta gcagcggtag cggtagtgag aacctgtatt tccaaggtga | 1020 |
| ttataaagat gacgatgata agtggtccca tccgcagttt gaaaaaagcc gttaaggtac | 1080 |
| cactcgagta aggatctcca ggcatcaaat aaaacgaaag gctcagtcga aagactgggc | 1140 |
| ctttcgtttt atctgttgtt tgtcggtgaa cgctctctac tagagtcaca ctggctcacc | 1200 |
| ttcgggtggg cctttctgcg tttataccta gggtacgggt tttgctgccc gcaaacgggc | 1260 |
| tgttctggtg ttgctagttt gttatcagaa tcgcagatcc ggcttcagcc ggtttgccgg | 1320 |
| ctgaaagcgc tatttcttcc agaattgcca tgattttttc cccacgggag gcgtcactgg | 1380 |
| ctcccgtgtt gtcggcagct ttgattcgat aagcagcatc gcctgtttca ggctgtctat | 1440 |
| gtgtgactgt tgagctgtaa caagttgtct caggtgttca atttcatgtt ctagttgctt | 1500 |
| tgttttactg gtttcacctg ttctattagg tgttacatgc tgttcatctg ttacattgtc | 1560 |
| gatctgttca tggtgaacag ctttgaatgc accaaaaact cgtaaaagct ctgatgtatc | 1620 |
| tatctttttt acaccgtttt catctgtgca tatggacagt tttccctttg atatgtaacg | 1680 |
| gtgaacagtt gttctacttt tgtttgttag tcttgatgct tcactgatag atacaagagc | 1740 |
| cataagaacc tcagatcctt ccgtatttag ccagtatgtt ctctagtgtg gttcgttgtt | 1800 |
| tttgcgtgag ccatgagaac gaaccattga gatcatactt actttgcatg tcactcaaaa | 1860 |
| attttgcctc aaaactggtg agctgaattt ttgcagttaa agcatcgtgt agtgtttttc | 1920 |
| ttagtccgtt atgtaggtag gaatctgatg taatggttgt tggtattttg tcaccattca | 1980 |
| tttttatctg gttgttctca agttcggtta cgagatccat ttgtctatct agttcaactt | 2040 |
| ggaaaatcaa cgtatcagtc gggcggcctc gcttatcaac caccaatttc atattgctgt | 2100 |
| aagtgtttaa atctttactt attggtttca aaacccattg gttaagcctt ttaaactcat | 2160 |
| ggtagttatt ttcaagcatt aacatgaact taaattcatc aaggctaatc tctatatttg | 2220 |
| ccttgtgagt tttcttttgt gttagttctt ttaataacca ctcataaatc ctcatagagt | 2280 |
| atttgttttc aaaagactta acatgttcca gattatattt tatgaatttt tttaactgga | 2340 |
| aaagataagg caatatctct tcactaaaaa ctaattctaa tttttcgctt gagaacttgg | 2400 |
| catagtttgt ccactggaaa atctcaaagc ctttaaccaa aggattcctg atttccacag | 2460 |
| ttctcgtcat cagctctctg gttgctttag ctaatacacc ataagcattt tccctactga | 2520 |
| tgttcatcat ctgagcgtat tggttataag tgaacgatac cgtccgttct ttccttgtag | 2580 |
| ggttttcaat cgtggggttg agtagtgcca cacagcataa aattagcttg gtttcatgct | 2640 |
| ccgttaagtc atagcgacta atcgctagtt catttgcttt gaaaacaact aattcagaca | 2700 |
| tacatctcaa ttggtctagg tgattttaat cactatacca attgagatgg gctagtcaat | 2760 |
| gataattact agtccttttc ccgggtgatc tgggtatctg taaattctgc tagacctttg | 2820 |
| ctggaaaact tgtaaattct gctagaccct ctgtaaattc cgctagacct ttgtgtgttt | 2880 |
| tttttgttta tattcaagtg gttataattt atagaataaa gaaagaataa aaaaagataa | 2940 |
| aaagaataga tcccagccct gtgtataact cactacttta gtcagttccg cagtattaca | 3000 |
| aaaggatgtc gcaaacgctg tttgctcctc tacaaaacag accttaaaac cctaaaggct | 3060 |
| taagtagcac cctcgcaagc tcgggcaaat cgctgaatat tccttttgtc tccgaccatc | 3120 |
| aggcacctga gtcgctgtct ttttcgtgac attcagttcg ctgcgctcac ggctctggca | 3180 |
| gtgaatgggg gtaaatggca ctacaggcgc cttttatgga ttcatgcaag gaaactaccc | 3240 |
| ataatacaag aaaagcccgt cacgggcttc tcagggcgtt ttatggcggg tctgctatgt | 3300 |
| ggtgctatct gactttttgc tgttcagcag ttcctgccct ctgattttcc agtctgacca | 3360 |
| cttcggatta tcccgtgaca ggtcattcag actggctaat gcacccagta aggcagcggt | 3420 |
| atcatcaaca ggcttacccg tcttactgtc cctagtgctt ggattctcac caataaaaaa | 3480 |
| cgcccggcgg caaccgagcg ttctgaacaa atccagatgg agttctgagg tcattactgg | 3540 |
| atctatcaac aggagtccaa gcgagctcga tatcaaatta cgccccgccc tgccactcat | 3600 |
| cgcagtactg ttgtaattca ttaagcattc tgccgacatg gaagccatca caaacggcat | 3660 |
| gatgaacctg aatcgccagc ggcatcagca ccttgtcgcc ttgcgtataa tatttgccca | 3720 |
| tggtgaaaac gggggcgaag aagttgtcca tattggccac gtttaaatca aaactggtga | 3780 |
| aactcaccca gggattggct gagacgaaaa acatattctc aataaaccct ttagggaaat | 3840 |
| aggccaggtt ttcaccgtaa cacgccacat cttgcgaata tatgtgtaga aactgccgga | 3900 |
| aatcgtcgtg gtattcactc cagagcgatg aaaacgtttc agtttgctca tggaaaacgg | 3960 |
| tgtaacaagg gtgaacacta tcccatatca ccagctcacc gtctttcatt gccatacgaa | 4020 |
| attccggatg agcattcatc aggcgggcaa gaatgtgaat aaaggccgga taaaacttgt | 4080 |
| gcttattttt ctttacggtc tttaaaaagg ccgtaatatc cagctgaacg gtctggttat | 4140 |
| aggtacattg agcaactgac tgaaatgcct caaaatgttc tttacgatgc cattgggata | 4200 |
| tatcaacggt ggtatatcca gtgatttttt tctccatttt agcttcctta gctcctgaaa | 4260 |
| atctcgataa ctcaaaaaat acgcccggta gtgatcttat ttcattatgg tgaaagttgg | 4320 |
| aacctcttac gtgccgatca acgtctcatt ttcgccagat atc | 4363 |
| SEQ ID NO: 11 | |
| gacgtcttga cagctagctc agtcctaggg attgtgctag caggtttaat cgaattgacg | 60 |
| tctttacggc tagctcagtc ctaggtacta tgctagcagg tttaatcgaa ttcaaaagat | 120 |
| cttttaagaa ggagatatac atatgattac tcatggttgt tatacccgga cccggcacaa | 180 |
| gcataagcta aaaaaaacat tgattatgct tagtgctggt ttaggattgt ttttttatgt | 240 |
| taatcagaac tcatttgcaa atggtgaaaa ttattttaaa ttgggttcgg attcaaaact | 300 |
| gttaactcat gatagctatc agaatcgcct tttttatacg ttgaaaactg gtgaaactgt | 360 |
| tgccgatctt tctaaatcgc aagatattaa tttatcgacg atttggtcgt tgaataagca | 420 |
| tttatacagt tctgaaagcg aaatgatgaa ggccgcgcct ggtcagcaga tcattttgcc | 480 |
| actcaaaaaa cttccctttg aatacagtgc actaccactt ttaggttcgg cacctcttgt | 540 |
| tgctgcgggt ggtgttgctg gtcacacgaa taaactgact aaaatgtccc cggacgtgac | 600 |
| caaaagcaac atgaccgatg acaaggcatt aaattatgcg gcacaacagg cggcgagtct | 660 |
| cggtagccag cttcagtcgc gatctctgaa cggcgattac gcgaaagata ccgctcttgg | 720 |
| tatcgctggt aaccaggctt cgtcacagtt gcaggcctgg ttacaacatt atggaacggc | 780 |
| agaggttaat ctgcaaagtg gtaataactt tgacggtagt tcactggact tcttattacc | 840 |
| gttctatgat tccgaaaaaa tgctggcatt tggtcaggtc ggagcgcgtt acattgactc | 900 |
| ccgctttacg gcaaatttag gtgcgggtca gcgttttttc cttcctgcaa acatgttggg | 960 |
| ctataacgtc ttcattgatc aggatttttc tggtgataat acccgtttag gtattggtgg | 1020 |
| cgaatactgg cgagactatt tcaaaagtag cgttaacggc tatttccgca tgagcggctg | 1080 |
| gcatgagtca tacaataaga aagactatga tgagcgccca gcaaatggct tcgatatccg | 1140 |
| ttttaatggc tatctaccgt catatccggc attaggcgcc aagctgatat atgagcagta | 1200 |
| ttatggtgat aatgttgctt tgtttaattc tgataagctg caatcgaatc ctggtgcggc | 1260 |
| gaccgttggt gtaaactata ctccgattcc tctggtgacg atggggatcg attaccgtca | 1320 |
| tggtacgggt aatgaaaatg atctccttta ctcaatgcag ttccgttatc agtttgataa | 1380 |
| atcgtggtct cagcaaattg aaccacagta tgttaacgag ttaagaacat tatcaggcag | 1440 |
| ccgttacgat ctggttcagc gtaataacaa tattattctg gagtacaaga agcaggatat | 1500 |
| tctttctctg aatattccgc atgatattaa tggtactgaa cacagtacgc agaagattca | 1560 |
| gttgatcgtt aagagcaaat acggtctgga tcgtatcgtc tgggatgata gtgcattacg | 1620 |
| cagtcagggc ggtcagattc agcatagcgg aagccaaagc gcacaagact accaggctat | 1680 |
| tttgcctgct tatgtgcaag gtggcagcaa tatttataaa gtgacggctc gcgcctatga | 1740 |
| ccgtaatggc aatagctcta acaatgtaca gcttactatt accgttctgt cgaatggtca | 1800 |
| agttgtcgac caggttgggg taacggactt tacggcggat aagacttcgg ctaaagcgga | 1860 |
| taacgccgat accattactt ataccgcgac ggtgaaaaag aatggggtag ctcaggctaa | 1920 |
| tgtccctgtt tcatttaata ttgtttcagg aactgcaact cttggggcaa atagtgccaa | 1980 |
| aacggatgct aacggtaagg caaccgtaac gttgaagtcg agtacgccag gacaggtcgt | 2040 |
| cgtgtctgct aaaaccgcgg agatgacttc agcacttaat gccagtgcgg ttatattttt | 2100 |
| tgatggtgcg cccgggaagc ttgtcgacgg agctcgataa tccggcaaaa aagggcaagg | 2160 |
| tgtcaccacc ctgccctttt tctttaaaac cgaaaagatt acttcgcgtt atgcaggctt | 2220 |
| cctcgctcac tgactcgctg cgctcggtcg ttcggctgcg gcgagcggta tcagctcact | 2280 |
| caaaggcggt aatctcgagt cgatccaaac tcgagtaagg atctccaggc atcaaataaa | 2340 |
| acgaaaggct cagtcgaaag actgggcctt tcgttttatc tgttgtttgt cggtgaacgc | 2400 |
| tctctactag agtcacactg gctcaccttc gggtgggcct ttctgcgttt atacctaggg | 2460 |
| tacgggtttt gctgcccgca aacgggctgt tctggtgttg ctagtttgtt atcagaatcg | 2520 |
| cagatccggc ttcagccggt ttgccggctg aaagcgctat ttcttccaga attgccatga | 2580 |
| ttttttcccc acgggaggcg tcactggctc ccgtgttgtc ggcagctttg attcgataag | 2640 |
| cagcatcgcc tgtttcaggc tgtctatgtg tgactgttga gctgtaacaa gttgtctcag | 2700 |
| gtgttcaatt tcatgttcta gttgctttgt tttactggtt tcacctgttc tattaggtgt | 2760 |
| tacatgctgt tcatctgtta cattgtcgat ctgttcatgg tgaacagctt tgaatgcacc | 2820 |
| aaaaactcgt aaaagctctg atgtatctat cttttttaca ccgttttcat ctgtgcatat | 2880 |
| ggacagtttt ccctttgata tgtaacggtg aacagttgtt ctacttttgt ttgttagtct | 2940 |
| tgatgcttca ctgatagata caagagccat aagaacctca gatccttccg tatttagcca | 3000 |
| gtatgttctc tagtgtggtt cgttgttttt gcgtgagcca tgagaacgaa ccattgagat | 3060 |
| catacttact ttgcatgtca ctcaaaaatt ttgcctcaaa actggtgagc tgaatttttg | 3120 |
| cagttaaagc atcgtgtagt gtttttctta gtccgttatg taggtaggaa tctgatgtaa | 3180 |
| tggttgttgg tattttgtca ccattcattt ttatctggtt gttctcaagt tcggttacga | 3240 |
| gatccatttg tctatctagt tcaacttgga aaatcaacgt atcagtcggg cggcctcgct | 3300 |
| tatcaaccac caatttcata ttgctgtaag tgtttaaatc tttacttatt ggtttcaaaa | 3360 |
| cccattggtt aagcctttta aactcatggt agttattttc aagcattaac atgaacttaa | 3420 |
| attcatcaag gctaatctct atatttgcct tgtgagtttt cttttgtgtt agttctttta | 3480 |
| ataaccactc ataaatcctc atagagtatt tgttttcaaa agacttaaca tgttccagat | 3540 |
| tatattttat gaattttttt aactggaaaa gataaggcaa tatctcttca ctaaaaacta | 3600 |
| attctaattt ttcgcttgag aacttggcat agtttgtcca ctggaaaatc tcaaagcctt | 3660 |
| taaccaaagg attcctgatt tccacagttc tcgtcatcag ctctctggtt gctttagcta | 3720 |
| atacaccata agcattttcc ctactgatgt tcatcatctg agcgtattgg ttataagtga | 3780 |
| acgataccgt ccgttctttc cttgtagggt tttcaatcgt ggggttgagt agtgccacac | 3840 |
| agcataaaat tagcttggtt tcatgctccg ttaagtcata gcgactaatc gctagttcat | 3900 |
| ttgctttgaa aacaactaat tcagacatac atctcaattg gtctaggtga ttttaatcac | 3960 |
| tataccaatt gagatgggct agtcaatgat aattactagt ccttttcccg ggtgatctgg | 4020 |
| gtatctgtaa attctgctag acctttgctg gaaaacttgt aaattctgct agaccctctg | 4080 |
| taaattccgc tagacctttg tgtgtttttt ttgtttatat tcaagtggtt ataatttata | 4140 |
| gaataaagaa agaataaaaa aagataaaaa gaatagatcc cagccctgtg tataactcac | 4200 |
| tactttagtc agttccgcag tattacaaaa ggatgtcgca aacgctgttt gctcctctac | 4260 |
| aaaacagacc ttaaaaccct aaaggcttaa gtagcaccct cgcaagctcg ggcaaatcgc | 4320 |
| tgaatattcc ttttgtctcc gaccatcagg cacctgagtc gctgtctttt tcgtgacatt | 4380 |
| cagttcgctg cgctcacggc tctggcagtg aatgggggta aatggcacta caggcgcctt | 4440 |
| ttatggattc atgcaaggaa actacccata atacaagaaa agcccgtcac gggcttctca | 4500 |
| gggcgtttta tggcgggtct gctatgtggt gctatctgac tttttgctgt tcagcagttc | 4560 |
| ctgccctctg attttccagt ctgaccactt cggattatcc cgtgacaggt cattcagact | 4620 |
| ggctaatgca cccagtaagg cagcggtatc atcaacaggc ttacccgtct tactgtccct | 4680 |
| agtgcttgga ttctcaccaa taaaaaacgc ccggcggcaa ccgagcgttc tgaacaaatc | 4740 |
| cagatggagt tctgaggtca ttactggatc tatcaacagg agtccaagcg agctcgatat | 4800 |
| caaattacgc cccgccctgc cactcatcgc agtactgttg taattcatta agcattctgc | 4860 |
| cgacatggaa gccatcacaa acggcatgat gaacctgaat cgccagcggc atcagcacct | 4920 |
| tgtcgccttg cgtataatat ttgcccatgg tgaaaacggg ggcgaagaag ttgtccatat | 4980 |
| tggccacgtt taaatcaaaa ctggtgaaac tcacccaggg attggctgag acgaaaaaca | 5040 |
| tattctcaat aaacccttta gggaaatagg ccaggttttc accgtaacac gccacatctt | 5100 |
| gcgaatatat gtgtagaaac tgccggaaat cgtcgtggta ttcactccag agcgatgaaa | 5160 |
| acgtttcagt ttgctcatgg aaaacggtgt aacaagggtg aacactatcc catatcacca | 5220 |
| gctcaccgtc tttcattgcc atacgaaatt ccggatgagc attcatcagg cgggcaagaa | 5280 |
| tgtgaataaa ggccggataa aacttgtgct tatttttctt tacggtcttt aaaaaggccg | 5340 |
| taatatccag ctgaacggtc tggttatagg tacattgagc aactgactga aatgcctcaa | 5400 |
| aatgttcttt acgatgccat tgggatatat caacggtggt atatccagtg atttttttct | 5460 |
| ccattttagc ttccttagct cctgaaaatc tcgataactc aaaaaatacg cccggtagtg | 5520 |
| atcttatttc attatggtga aagttggaac ctcttacgtg ccgatcaacg tctcattttc | 5580 |
| gccagatatc | 5590 |

What is claimed is:

1. An engineered probiotic bacterium comprising a heterologous nucleic acid, wherein the heterologous nucleic acid comprises a nucleic acid sequence encoding an anti-spike glycoprotein nanobody of a coronavirus.

2. The engineered probiotic bacterium of claim 1 wherein the bacterium is Escherichia coli Nissle 1917.

3. The engineered probiotic bacterium of claim 1 wherein the nucleic acid sequence is selected from the group consisting of SEQ ID NO: 1, SEQ ID NO: 2, SEQ ID NO: 3 and SEQ ID NO: 4.

4. The engineered probiotic bacterium of claim 1 wherein the anti-spike glycoprotein nanobody appears on the surface of the probiotic bacteria.

5. The engineered probiotic bacterium of claim 1 wherein the heterologous nucleic acid is located in a plasmid.

6. The engineered probiotic bacterium of claim 5 wherein the plasmid is selected from the group consisting of pNKLab001, pNKLab002, pNKLab003 and pNKLab004.

7. The engineered probiotic bacterium of claim 5 wherein the plasmid further comprises a surface display signal.

8. The engineered probiotic bacterium of claim 7 wherein the surface display signal is selected from the group consisting of Intimin and Lpp-OmpA.

9. The engineered probiotic bacterium of claim 5 wherein the plasmid has a nucleic acid sequence selected from the group consisting of SEQ ID NO: 5, SEQ ID NO: 6, SEQ ID NO: 7, SEQ ID NO: 8, SEQ ID NO: 9 and SEQ ID NO: 10.

10. The engineered probiotic bacterium of claim 5 wherein the plasmid is a CJ23 plasmid.

11. A pharmaceutical composition comprising the engineered probiotic bacterium of claim 1 and a pharmaceutically acceptable excipient.

12. A method for preventing a disease or disorder in a subject, the method comprising administering the engineered probiotic bacterium of claim 1 to the subject, wherein the engineered probiotic bacterium expresses an anti-spike glycoprotein nanobody, thereby preventing the disease or disorder in the subject.
